(19) United States
(12) Patent Application Publication
(10) Pub. No.: US 2026/0033772 A1
(43) Pub. Date: Feb. 5, 2026

(54) POSTURE DETECTION SYSTEM AND POSTURE DETECTION METHOD

(71) Applicant: NISHIKAWA Co., Ltd., Chuo-ku, Tokyo (JP)

(72) Inventors: Yasuyuki NISHIKAWA, Chuo-ku, Tokyo (JP); Takuto NONOMURA, Chuo-ku, Tokyo (JP); Hideki WADA, Chuo-ku, Tokyo (JP); Hideaki MOGI, Chuo-ku, Tokyo (JP); Sho IKEDA, Chuo-ku, Tokyo (JP)

(21) Appl. No.: 18/996,097

(22) PCT Filed: Jul. 21, 2023
(86) PCT No.: PCT/JP2023/026795

§ 371 Date: Jan. 17, 2025

(30) Foreign Application Priority Data
Jul. 25, 2022 (JP) 2022-118001

(51) Int. Cl. A61B 5/00; A47C 7/02

(52) U.S. Cl. CPC A61B 5/4561; A47C 7/02; A61B 5/6892; A61B 5/746; A61B 2562/0252

(57) ABSTRACT

A posture detection system that detects the posture of a user seated on a cushion that includes a seating portion having a buttock support portion and a lumbar support portion having a sacral support portion. The posture detection system includes a seating portion sensor attached to the seating portion and configured to measure whether or not the user is seated on the seating portion, a sacral support portion sensor attached to the sacral support portion and configured to measure the posture of the user seated on the seating portion, a posture determination unit configured to determine the posture of the user based on a result measured by the sacral support portion sensor, and an alarm unit configured to output an alarm if the posture determination unit determines that the posture of the user is unsatisfactory.

### TECHNICAL FIELD

The present disclosure relates to a posture detection system and a posture detection method of detecting the posture of a user seated on a cushion.

### BACKGROUND ART

Japanese Unexamined Patent Publication No. H6-86725 discloses a seat device. The seat device includes a seat cushion that supports the buttocks of a seated person with a seat surface, a seat back that supports the back of the seated person with a support surface, an actuator that drives to change the shape of the support surface of the seat back, and a detection means that extracts an element related to the physical figure characteristics of the upper half of the body of the seated person.

The actuator is an airbag provided inside the seat back. The detection means is a seat pressure sensor provided inside both the seat cushion and the seat back. The detection means extracts the physical figure characteristics of the upper half of the body of the seated person from detection values obtained by each seat pressure sensor. The actuator inflates the airbag in response to the extracted physical figure characteristics to control the posture of the seated person.

Japanese Unexamined Patent Publication Nos. 2019-151251 and 2021-112665 disclose a seat. The seat includes a first cushion sensor arranged at a position corresponding to the buttocks of the seated occupant and a second cushion sensor located in front of the first cushion sensor.

The seat further includes a first back sensor arranged at a lower part of the seat back, a second back sensor arranged above the first back sensor, and a control unit. The control unit identifies the movement of the seated person based on the outputs of at least two of the first cushion sensor, the second cushion sensor, the first back sensor, and the second back sensor.

### CITATION LIST

### Patent Literature

Patent Literature 1: Japanese Unexamined Patent Publication No. H6-86725
Patent Literature 2: Japanese Unexamined Patent Publication No. 2019-151251
Patent Literature 3: Japanese Unexamined Patent Publication No. 2021-112665

### SUMMARY OF INVENTION

The posture detection system according to the present disclosure is (1) a posture detection system provided for detecting the posture of a user seated on a cushion that includes a seating portion having a buttock support portion on which a buttock of the user is placed and a lumbar support portion having a sacral support portion configured to support a sacrum of the user from behind. The posture detection system includes a seating portion sensor attached to the seating portion and configured to measure whether or not the user is seated on the seating portion, a sacral support portion sensor attached to the sacral support portion and configured to measure the posture of the user seated on the seating portion, a posture determination unit configured to determine the posture of the user based on a result measured by the sacral support portion sensor, and an alarm unit configured to output an alarm if the posture determination unit determines that the posture of the user is unsatisfactory.

In the posture detection system, the cushion includes the seating portion having the buttock support portion that supports the user's buttock, and a sacral support portion that supports the user's sacrum from behind. The posture detection system includes the seating portion sensor that measures whether or not the user is seated on the seating portion of the cushion. The measurement of whether or not the user is seated on the seating portion by the seating portion sensor makes it possible to calculate the time period during which the user is seated on the seating portion. The user can grasp whether or not a long period of sitting has occurred, thereby avoiding issues such as impaired circulation in the legs and lower back or a decline in muscle strength. The posture detection system includes sacral support portion sensor that is provided in the sacral support portion and measures the posture of the user seated on the cushion. The sacrum is a part that protrudes from the user's back. The sacral support portion sensor being provided in the sacral support portion that supports the sacrum enables the posture of the user to be measured with high accuracy. The posture detection system includes the alarm unit that outputs an alarm if the posture determination unit determines that the posture is unsatisfactory. The alarm unit that outputs an alarm if the posture is poor enables the user to grasp that the posture is improper, thereby reducing the possibility of injury to the body due to unsatisfactory posture.

(2) In the above (1), the sacral support portion sensor may measure whether or not the user's sacrum is in contact with the sacral support portion sensor. The posture determination unit may determine that the user's posture is hunched if the user's sacrum is not in contact with the sacral support portion sensor. In this case, the posture determination unit determines that the user is hunched if the user leans forward and the sacrum is not in contact with the sacral support portion sensor. The determination of whether or not the user is hunched based on the presence or absence of contact with the sacral support portion sensor enables the user's posture to be grasped with high accuracy, suppressing hunched posture.

(3) In the above (1) or (2), the sacral support portion sensor may measure the load of the user's sacrum on the sacral support portion sensor. The posture determination unit may determine that the user's posture is excessively leaning backward if the load measured by the sacral support portion sensor is equal to or greater than a predetermined value. In this case, the posture determination unit determines that the user is excessively leaning backward if the load on the sacral support portion sensor is equal to or greater than the predetermined value. The determination of whether or not the user is excessively leaning backward based on the load on the sacral support portion sensor makes it possible to suppress a decrease in abdominal muscle strength due to excessive leaning backward.

(4) In any of the above (1) to (3), the seating portion may have a pair of thigh support portions aligned along the left-to-right direction as viewed by the user seated on the seating portion. The posture detection system may further include a pair of thigh support portion sensors attached to the pair of thigh support portions, respectively, to measure the load on the thighs of the user seated on the seating portion. In this case, the measurement of the load on each of the user's left and right thighs by the pair of thigh support portion sensors enables the left-to-right balance of the thighs to be grasped. Furthermore, if one of the paired thigh support portion sensors detects a load and the other of the paired thigh support portion sensors does not detect a load, it can be determined that the user's legs are likely to be crossed. Thus, the posture of the user seated on the cushion can be grasped with higher accuracy.

(5) In any of the above (1) to (4), the posture detection system may include a fabric to which the seating portion sensor and the sacral support portion sensor are attached and which covers at least a part of the core material of the cushion. In this case, the seating portion sensor and the sacral support portion sensor are attached to the fabric, and at least a part of the core material is covered by this fabric. Since the seating portion sensor and the sacral support portion sensor can be attached to the cushion by covering the core material of the existing cushion with the fabric, the seating portion sensor and the sacral support portion sensor can be easily arranged on the existing cushion.

(6) In the above (5), the posture detection system may include an outer fabric covering the core material, and the fabric may be an inner fabric covered by the outer fabric. In this case, the outer fabric is provided on the outside of the inner fabric to which the seating portion sensor and the sacral support portion sensor are attached. Since the outer fabric can be removed and washed, the cushion can be maintained in a more hygienic condition, allowing for prolonged use of the cushion.

(7) In the above (4), the core material of the cushion may include a concave portion formed on the upper surface of the seating portion and a concave portion formed on the upper surface of the thigh support portion. The seating portion sensor may be embedded in the concave portion of the seating portion, and the thigh support portion sensor may be embedded in the concave portion of the thigh support portion.

(8) In any of the above (1) to (5), the sacral support portion may include a protruding surface with a forward-protruding curved shape and a recessed portion that is indented rearward from the protruding surface, and the sacral support portion sensor may be attached to the recessed portion of the sacral support portion.

(9) In the above (4), the posture detection system may include a fabric that covers at least a part of the core material of the cushion. The fabric may be an inner fabric to which the seating portion sensor, the sacral support portion sensor, and the thigh support portion sensor are attached.

(10) In the above (5), the fabric may cover only a part of the core material. The fabric may be fixed to the core material in such a way as to cover the seating portion and the sacral support portion.

(11) In the above (4), the seating portion may extend in a first direction, which is a front-to-rear direction as viewed by the user seated on the seating portion, and in a second direction, which is a left-to-right direction as viewed by the user seated on the seating portion. The posture detection system may include two seating portion sensors aligned along the second direction, one sacral support portion sensor, and two thigh support portion sensors aligned along the second direction.

(12) In any of the above (1) to (11), the buttock support portion may include a pair of ischial support portions that support the ischium of the user seated on the seating portion. The seating portion sensor may be arranged around each of the paired ischial support portions.

(13) In any of the above (1) to (12), the posture detection system may include a leg-crossing determination unit that determines whether or not the user's legs seated on the seating portion are crossed.

(14) In the above (3), the posture determination unit may determine that the user's posture is proper if the load measured by the sacral support portion sensor is less than the predetermined value.

(15) In the above (4), the posture determination unit may calculate the difference in loads applied to the pair of thigh support portion sensors as measured by the thigh support portion sensors.

(16) In the above (4), the posture determination unit may calculate the absolute value of the difference between the load on one of the thigh support portion sensors received from the left thigh and the load on the other thigh support portion sensor received from the right thigh, determine that the left-to-right balance is unsatisfactory and the posture is improper if the calculated absolute value is equal to or greater than a predetermined value, and determine that the left-to-right balance is satisfactory if the calculated absolute value is not equal to or greater than the predetermined value.

(17) In the above (4), the posture detection system may include a leg-crossing determination unit that determines whether or not legs of the user seated on the seating portion are crossed, and the leg-crossing determination unit may determine that the user's legs are crossed if one of the paired thigh support portion sensors receives a load from the thigh and the other thigh support portion sensor does not receive a load from the thigh.

(18) In any of the above (1) to (17), the posture detection system may include a seating determination unit that determines whether or not the user is seated on the seating portion, and a display unit that displays the result determined by each of the seating determination unit and the posture determination unit. The display unit may display, as a time chart, at least one of a time period during which the user is seated on the seating portion, a time period during which the posture is proper, a time period during which the user is hunched, a time period during which the user is excessively leaning backward, a time period during which the left-to-right balance is unsatisfactory, and a time period during which the user's legs are crossed.

(19) In any of the above (1) to (18), the posture detection system may include a seating determination unit that determines whether or not the user is seated on the seating portion, and a display unit that displays the result determined by each of the seating determination unit and the posture determination unit. The display unit may display, as a comment, the result determined by the seating determination unit and the result determined by the posture determination unit.

The posture detection method according to the present disclosure is (20) a posture detection method that detects the posture of a user seated on a cushion that includes a seating portion having a buttock support portion on which the user's buttocks are placed, and a lumbar support portion having a sacral support portion that supports the user's sacrum from behind. The posture detection method includes steps of measuring whether or not the user is seated on the seating portion by a seating portion sensor attached to the seating portion, measuring the posture of the user seated on the seating portion by a sacral support portion sensor attached to the sacral support portion, determining the posture of the user based on a result measured by the sacral support portion sensor, and outputting an alarm if it is determined that the posture of the user is unsatisfactory in the determining step.

This posture detection method includes a step of measuring whether or not the user is seated on the seating portion of the cushion. The measurement of whether or not the user is seated on the seating portion with the seating portion sensor makes it possible to calculate the time period during which the user is seated on the seating portion, allowing for grasping whether or not a long period of sitting has occurred. Thus, it is possible to avoid issues such as impaired blood circulation in the legs and lower back and a decline in muscle strength. The posture detection method includes a step of measuring the posture of the user seated on the cushion by the sacral support portion sensor. The sacrum is a part that protrudes from the user's back. The measurement of the user's posture by the sacral support portion sensor provided in the sacral support portion that supports the sacrum allows the user's posture to be measured with high accuracy. The posture detection system includes a step of outputting an alarm if the posture determination unit determines that the posture is unsatisfactory. The alarm unit that outputs an alarm if the posture is poor enables the user to grasp that the posture is improper, thereby reducing the possibility of injury to the body due to unsatisfactory posture.

### BRIEF DESCRIPTION OF DRAWINGS

FIG. 1 is a perspective view illustrating a core material of a cushion used in a posture detection system according to an embodiment.

FIG. 2 is a plan view illustrating the core material of FIG. 1.

FIG. 3 is a bottom view illustrating the core material of FIG. 1.

FIG. 4 is a cross-sectional view taken along line A-A of FIG. 2.

FIG. 5 is a side view illustrating the core material of FIG. 1.

FIG. 6 is a schematic cross-sectional view illustrating a seating portion sensor, a sacral support portion sensor, a thigh support portion sensor, and a fabric of the posture detection system according to an embodiment.

FIG. 7 is a functional block diagram of the posture detection system according to an embodiment.

FIG. 8 is a view illustrating an exemplary display screen by a display unit of the posture detection system of FIG. 7.

FIG. 9 is a schematic cross-sectional view illustrating a seating portion sensor, a sacral support portion sensor, a thigh support portion sensor, and a fabric of a posture detection system according to a modification.

FIG. 10 is a perspective view illustrating a lumbar support and a cushion to which the posture detection system and the posture detection method of the embodiment can be applied.

FIG. 11 is a plan view illustrating the lumbar support and the cushion of FIG. 10.

FIG. 12 is a perspective view of the lumbar support and cushion of FIG. 10 as viewed from the rear.

FIG. 13 is a perspective view of the lumbar support of FIG. 10.

FIG. 14 is a plan view illustrated to describe the operation of the lumbar support of FIG. 13.

### DESCRIPTION OF EMBODIMENTS

For example, in office environments, people may spend long hours working while seated on a cushion placed on the seat of a chair. In such cases, there is a possibility that the user who sits on the cushion will injure the body if the user's posture deteriorates. Thus, it is necessary to be able to detect the posture of the user seated on the cushion with high accuracy. Prolonged sitting can lead to issues such as impaired blood circulation in the lower back and legs, resulting in decreased muscle strength. Thus, it is required to be able to grasp how long a user remains seated on a cushion.

The present embodiment is intended to provide a posture detection system and a posture detection method capable of grasping the posture of a user seated on a cushion with high accuracy and grasping how long a user remains seated on a cushion.

The following describes an embodiment of the posture detection system and the posture detection method according to the present disclosure with reference to the drawings. In the description of the drawings, identical or equivalent elements are assigned the same reference numerals, and duplicated descriptions will be omitted as appropriate. The drawings may be simplified or exaggerated in some parts for ease of understanding, and the dimensional ratios and the like are not limited to those illustrated in the drawings.

In the embodiment, the posture detection system detects the posture of a user seated on a cushion. The cushion is constituted by a flexible material. The term “flexible material” refers to a cushioning material that deforms in response to the load applied by a person's body placed on it. The cushion includes a seating portion having a buttock support portion on which the user's buttocks are placed and includes a lumbar support portion having a sacral support portion that supports the user's sacrum from behind.

The term “user” refers to a user of the cushion, for example, a person seated on the cushion. The term “buttocks” refers to the rounded lower part of the body, located below the waist and above the thighs. The term “sitting” or “seated” refers to sitting on the cushion. The term “seating portion” refers to the part of the cushion that comes into contact with the buttocks in sitting on the cushion. The term “thigh” refers to the part of the legs from the base of the legs to the knees. The buttock support portion has, for example, a pair of ischial support portions. The term “ischium” or “ischial” refers to the part of the pelvis that is located at the lowest part as the user sits on the seating portion.

The term “posture” refers to the physical condition of the user seated on the cushion. The posture detection system according to the embodiment outputs an alarm if the user's posture is unsatisfactory. The term “unsatisfactory posture” refers to at least one of the conditions including, for example, the user's back is hunched in sitting on the seating portion, the user's back is excessively leaning backward too far in sitting on the seating portion, and the user's legs are crossed.

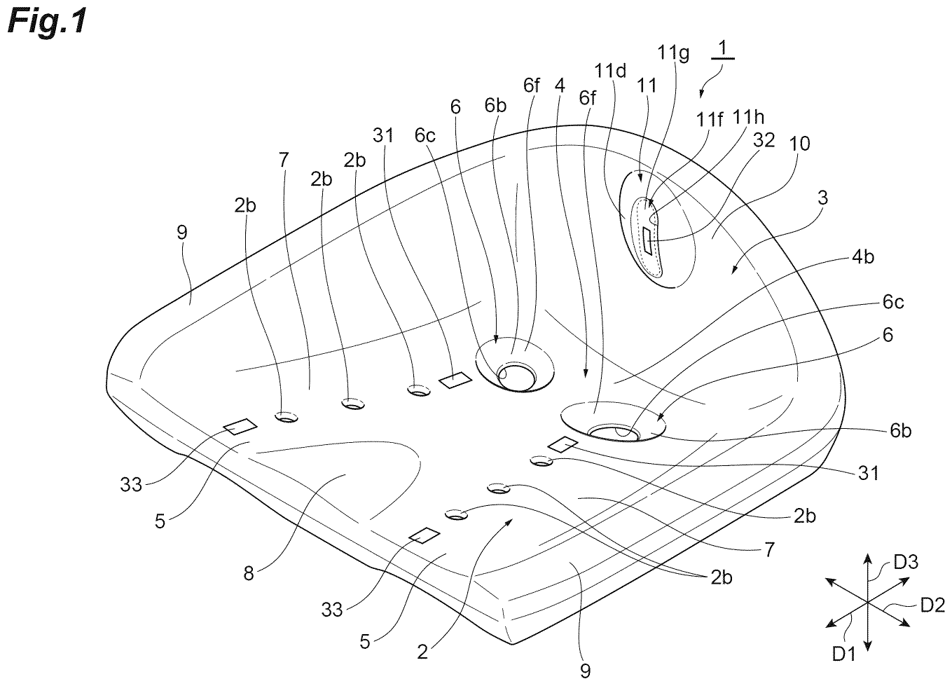

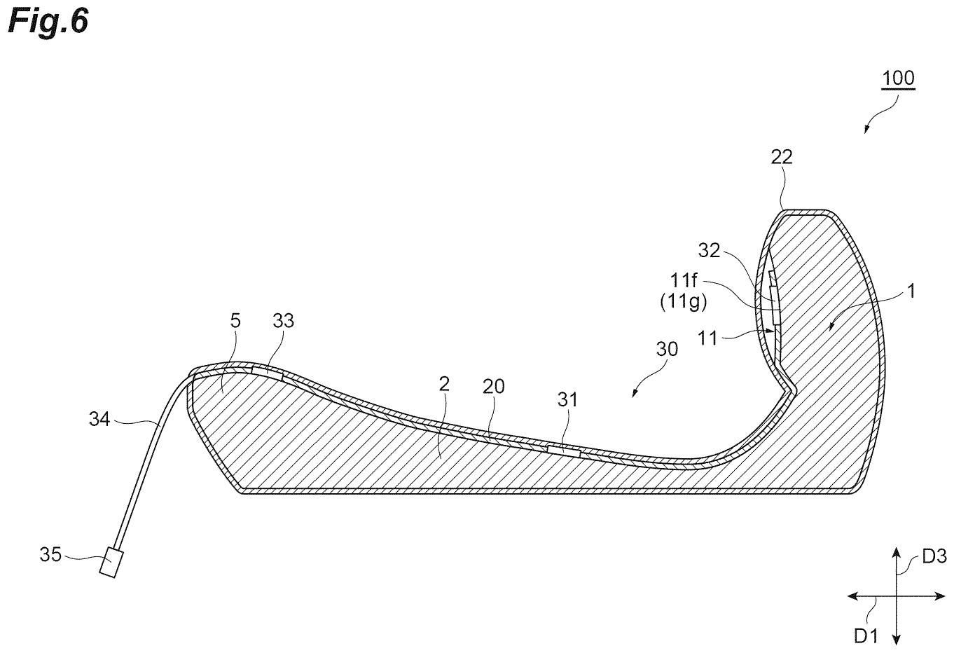

FIG. 1 is a perspective view illustrating a core material 1 of a cushion 100 according to the embodiment (see FIG. 6). As illustrated in FIG. 1, the core material 1 includes a seating portion 2 that extends horizontally and a lumbar support portion 3 that extends upward from the seating portion 2. The core material 1 is constituted by a flexible material. For example, the core material 1 is constituted by polyurethane foam.

In one example, the core material 1 is formed by urethane molding. In the case where the core material 1 is constituted by polyurethane foam and formed by urethane molding, the core material 1 constituted by a flexible material can be easily manufactured. The core material 1 is, for example, housed in a fabric 20 of the cushion 100 (see FIG. 6) for use.

Moreover, the above flexible material may include, for example, a thermoplastic elastomer. The flexible material may be a material with elasticity sufficient to deform in response to the load applied by a person's body placed on it. The flexible material concerned may be a resin (e.g., polypropylene, polyester, or polyethylene) that deforms in response to the load applied by a person's body placed on it. The type of the concerned flexible material is modifiable as appropriate.

The seating portion 2 extends in a first direction D1 and a second direction D2 that intersects with the first direction D1. The first direction D1 is the front-to-rear direction as viewed from the user seated on the seating portion 2, and the second direction D2 is the left-to-right direction as viewed from the user seated on the seating portion 2. The seating portion 2 has a thickness in a third direction D3 that intersects both the first direction D1 and the second direction D2. For example, the third direction D3 is the vertical direction.

In the following description, the forward direction as viewed from the user seated on the seating portion 2 may be referred to as “front”, “front side”, or “forward”, while the opposite direction to the forward direction may be referred to as “rear”, “rear side”, or “backward”. However, these directions are for convenience of description and are not intended to limit the position, orientation, or other aspects of each component.

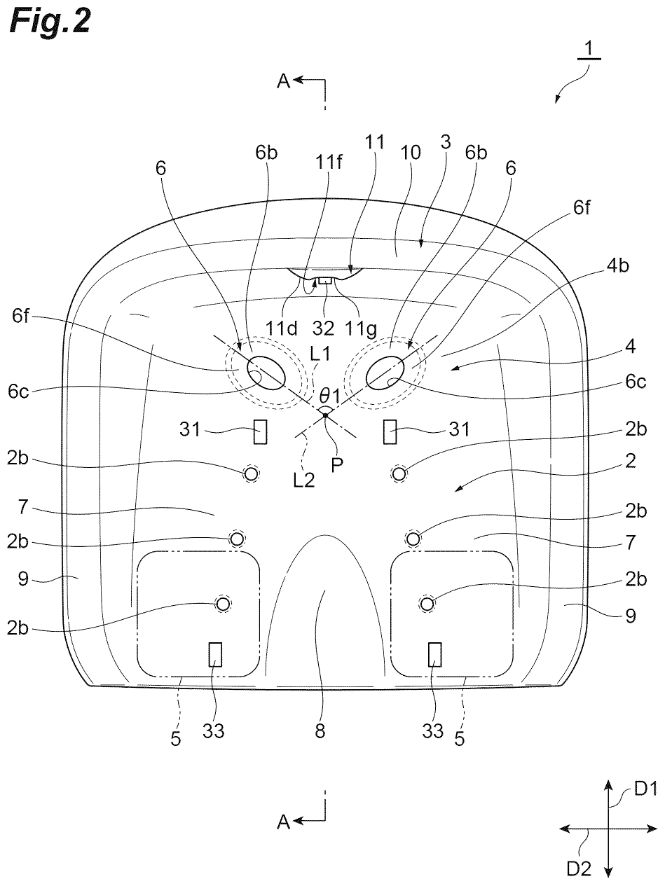

FIG. 2 is a plan view of the core material 1. As illustrated in FIGS. 1 and 2, the seating portion 2 has a buttock support portion 4 on which the user's buttocks are placed and has a thigh support portion 5 on which the user's thighs are placed. The buttock support portion 4 and the thigh support portion 5 are arranged to be aligned along the first direction D1. The buttock support portion 4 has a pair of ischial support portions 6 that support the ischium of a user seated on the seating portion 2.

For example, the seating portion 2 has a ventilation hole 2b. The ventilation hole 2b penetrates downward from an upper surface 4b of the buttock support portion 4 or an upper surface of the thigh support portion 5. The upper end of an inner wall defining the ventilation hole 2b may be R-chamfered to be rounded. In a plan view, a plurality of the ventilation holes 2b is arranged to align from the rear to the front.

For example, in a plan view (a plan view in which the lumbar support portion 3 is oriented to be located above the seating portion 2), a plurality (e.g., six) of the ventilation holes 2b may be arranged on both sides (left and right sides) in the second direction D2. For example, three ventilation holes 2b are arranged on each side in the second direction D2.

For example, two ventilation holes 2b are arranged along the second direction D2. The distance between the two ventilation holes 2b arranged along the second direction D2 increases, for example, from the buttock support portion 4 toward the thigh support portion 5 (in the forward direction). In one example, the multiple ventilation holes 2b are arranged in such a manner that they form a V-shape in a plan view.

For example, the core material 1 includes the ischial support portion 6 (e.g., a through-hole 6c described later) formed by urethane molding and includes the ventilation hole 2b formed by punching. In this case, the part of the mold used to manufacture the core material 1 that forms the ventilation hole 2b can be eliminated if not needed, so the shape of the mold can be simplified. Furthermore, in molding liquid urethane using the mold, an escape route for air in the liquid urethane can be secured, ensuring a clean finish for the core material 1.

The ischial support portion 6 is formed on the upper surface 4b of the buttock support portion 4 and is recessed relative to the upper surface 4b. The buttock support portion 4 has the paired ischial support portions 6 arranged along the second direction D2. The ischial support portion 6 has a concave portion 6b that is indented from the upper surface 4b of the buttock support portion 4. The ischial support portion 6 has the shape formed to have a major axis and a minor axis in a plan view. For example, the ischial support portion 6 has an oval shape (such as an ellipse shape) in a plan view. However, the shape of the ischial support portion 6 in a plan view is not limited to an oval or ellipse shape and also may be rectangular, rounded-rectangular, rhombic, or rounded-rhombic, and it can be appropriately modified as needed.

In the paired ischial support portions 6, an extension line L1 of the major axis of one of the paired ischial support portions 6 intersects with an extension line L2 of the major axis of the other of the paired ischial support portions 6. The extension line L1 and the extension line L2 have an intersection angle θ1 of, for example, 40° or more and 150° or less. The intersection angle θ1 may be 50° or more, 60° or more, 75° or more, or 90° or more. The intersection angle θ1 may be 135° or less, 120° or less, or 110° or less. In one example, the intersection angle θ1 is 100°.

The extension line L1 and the extension line L2 have an intersection point P located closer to the thigh support portion 5 (closer to the front) than the pair of ischial support portions 6. The intersection point P is located closer to the thigh support portion 5 than the center of each ischial support portion 6 in a plan view. The intersection point P is located on the side opposite to the lumbar support portion 3 as viewed from the pair of ischial support portions 6. The result allows the pair of ischial support portions 6 to form an inverted V shape in a plan view (plan view in the case where the lumbar support portion 3 is oriented to be located above the seating portion 2).

The distance between the centers of the paired ischial support portions 6 (the center-to-center distance of the paired ischial support portions 6) is, for example, 7 cm or more and 15 cm or less. In one example, the distance is 10 cm. The distance from the lumbar support portion 3 to the center of the ischial support portion 6 is, for example, 5 cm or more and 15 cm or less. In one example, the distance from the lumbar support portion 3 to the center of the ischial support portion 6 is 10 cm. In the case where each length and distance falls within the above value ranges, the user's ischium can be more appropriately fitted to each of the paired ischial support portions 6.

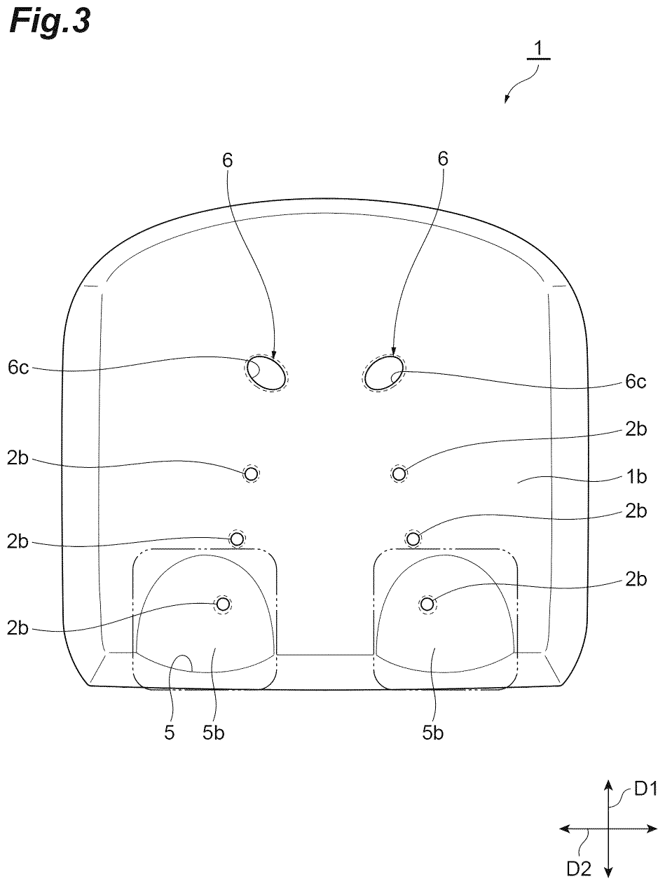

For example, each of the ischial support portions 6 has a through-hole 6c that penetrates downward from the upper surface 4b of the buttock support portion 4. Each of the ischial support portions 6 has an inner wall that defines the through-hole 6c and a tapered surface 6f located between the inner wall and the upper surface 4b. FIG. 3 is a bottom view illustrating the core material 1. As illustrated in FIGS. 2 and 3, the area of the ischial support portion 6 as viewed from above is larger than the area of the ischial support portion 6 as viewed from below.

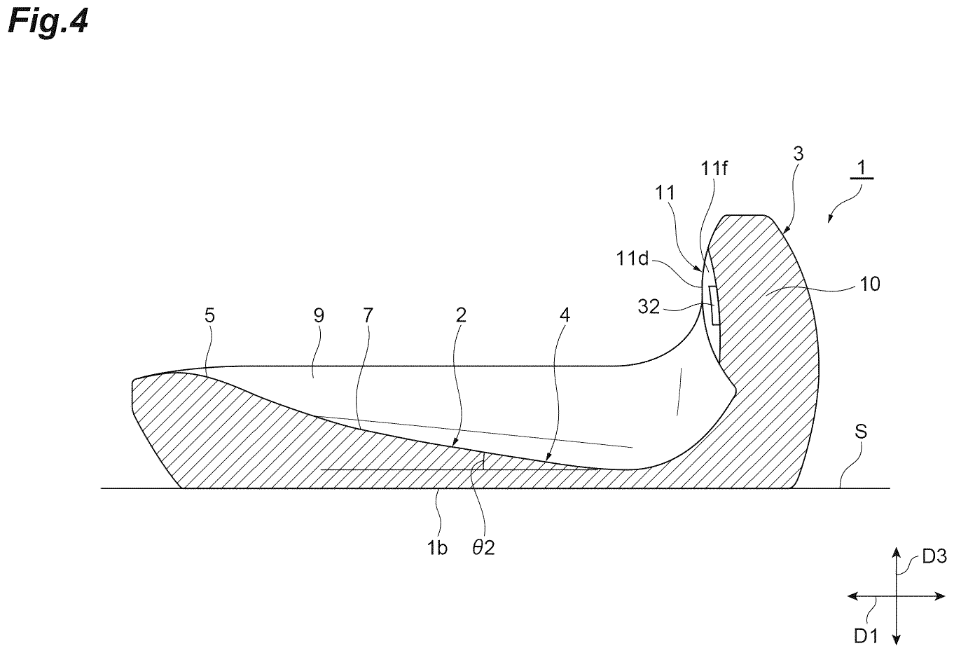

The core material 1 has a lower surface 1b that contacts a mounting surface S (see FIG. 4) on which the core material 1 is placed. For example, the inner wall defining the through-hole 6c connects the lower surface 1b and the tapered surface 6f to each other. At least one of the end of the inner wall of the through-hole 6c on the side of the lower surface 1b and the end of the inner wall on the side of the tapered surface 6f may be R-chamfered to be rounded.

The tapered surface 6f forms a concave portion 6b that is indented relative to the upper surface 4b. The tapered surface 6f is inclined in such a manner that the opening end of the tapered surface 6f expands in diameter as it approaches the upper surface 4b. For example, the area of the tapered surface 6f in a plan view is larger than the area of the through-hole 6c in a plan view. The tapered surface 6f may include a curved surface that is a R-chamfered portion in such a manner that the open end of the tapered surface 6f is rounded.

FIG. 4 is a cross-sectional view of line A-A in FIG. 2. As illustrated in FIGS. 2 and 4, the seating portion 2 has an inclined surface 7 that extends obliquely from the buttock support portion 4 toward the thigh support portion 5. For example, the seating portion 2 has the paired inclined surfaces 7 aligned along the second direction D2. For example, the inclined surface 7 is linear in a cross-section along a plane extending in the first direction D1 and the third direction D3. An inclination angle θ2 of the inclined surface 7 relative to the lower surface 1b of the core material 1 is, for example, 5° or more and 15° or less.

In the case where the inclination angle θ2 is 5° or more and 15° or less, the ischium of a user seated on the seating portion 2 can be made less likely to shift forward. Moreover, unlike the example mentioned above, the inclined surface 7 may have a curved shape (e.g., curved shape that curves so that the inclination angle θ2 increases toward the thigh support portion 5) in a cross-section along a plane extending in the first direction D1 and the third direction D3.

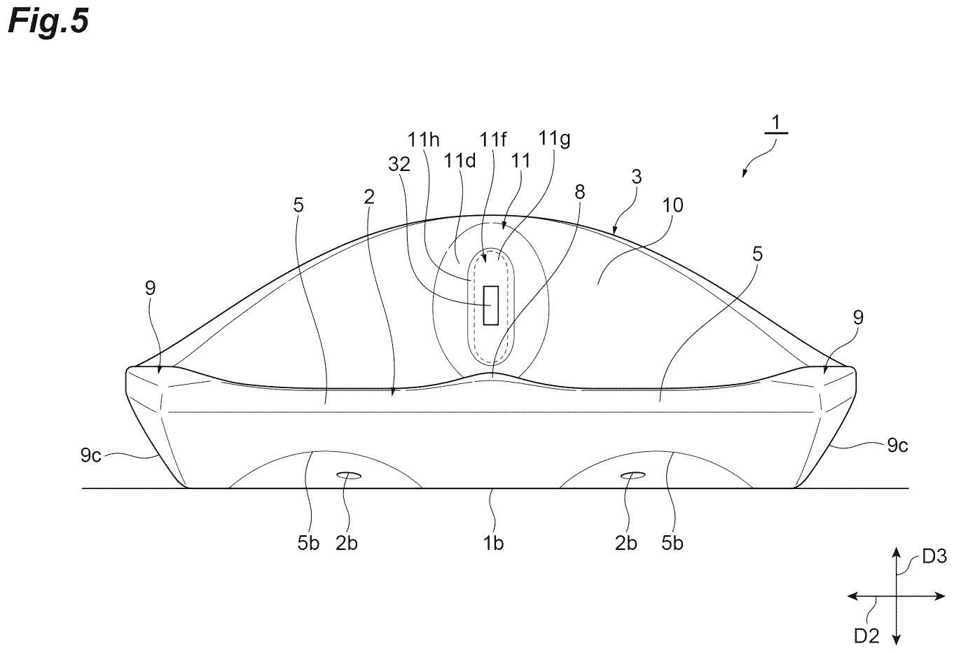

FIG. 5 is a side view of the core material 1 as viewed from the front. As illustrated in FIGS. 2, 4, and 5, the seating portion 2 has the paired thigh support portions 5 aligned along the second direction D2 and a convex portion 8 located between the pair of thigh support portions 5. Each of the paired thigh support portions 5 is located on the front side of the inclined surface 7.

The thigh support portion 5 provides a surface for the undersides of the user's thighs to rest on. In other words, the thigh support portion 5 supports the undersides of the user's thighs. As illustrated in FIGS. 3 and 5, the thigh support portion 5 has a concave portion 5b that is indented in the thickness direction (third direction D3) of the core material 1. The concave portion 5b is formed on the lower surface 1b of the core material 1.

The core material 1 has the paired concave portions 5b. For example, the paired concave portions 5b are provided at positions spaced apart from each other. The depth of the concave portion 5b becomes deeper as it moves further away from the lumbar support portion 3 (toward the front). For example, the thigh support portion 5 has the concave portion 5b that defines a space between itself and the mounting surface S on which the core material 1 is placed. In one example, the space defined between the concave portion 5b and the mounting surface S is formed to expand as it extends toward the front.

The convex portion 8 indicates a portion that protrudes from the inclined surface 7. The width of the convex portion 8 in a plan view becomes wider as it extends toward the front. For example, the shape of the convex portion 8 in a plan view is a parabola that expands as it moves forward. The height of the convex portion 8 increases as it approaches the center of the core material 1 in the second direction D2.

For example, the seating portion 2 has a pair of convex portions 9 located at both ends of the inclined surface 7 in the second direction D2. The convex portion 9 is a portion that connects the lumbar support portion 3 and the thigh support portion 5 to each other. The convex portion 9 extends along the first direction D1. Providing the above-mentioned convex portion 8 and convex portion 9 allows the thigh support portion 5 to be formed at each position on both sides of the convex portion 8 in the second direction D2 between the pair of convex portions 9. The result makes it possible to easily place the user's thigh on the thigh support portion 5.

The core material 1 has an inclined portion 9c that slopes toward the center of the core material 1 in the second direction D2 as it moves from the convex portion 9 toward the lower surface 1b. The core material 1 has, for example, the paired inclined portions 9c aligned along the second direction D2. In one example, the core material 1 has a boat-like shape that protrudes outward as it moves away from the lower surface 1b.

The lumbar support portion 3 extends upward from the end (rear end) of the seating portion 2 in the first direction D1. The lumbar support portion 3 has, for example, a general portion 10 that extends in both the second direction D2 and the third direction D3, and a sacral support portion 11 located at the center of the general portion 10 in the second direction D2. The general portion 10 refers to portions of the lumbar support portion 3 other than the sacral support portion 11.

As illustrated in FIGS. 2, 4, and 5, the sacral support portion 11 has a convex shape that protrudes forward. The sacral support portion 11 protrudes forward from the general portion 10. In one example, the shape of the sacral support portion 11 as viewed from the front has a shape with a major axis and a minor axis. For example, the shape of the sacral support portion 11 as viewed from the front is an oval shape (e.g., an ellipse shape).

For example, the sacral support portion 11 has a protruding surface 11d with a forward-protruding curved shape. The height of the protruding surface 11d from the general portion 10 increases from the outer edge of the protruding surface 11d toward the inside of the protruding surface 11d. The sacral support portion 11 may also have a recessed portion 11f that is indented backward from the protruding surface 11d. The recessed portion 11f is a part into which the sacrum of the user of the cushion 100 fits.

The recessed portion 11f extends in both the second direction D2 and the third direction D3. For example, the length of the recessed portion 11f in the third direction D3 is longer than the length (width) of the recessed portion 11f in the second direction D2. The shape of the recessed portion 11f as viewed from the first direction D1 may be a groove shape (or an elliptical shape) extending in the third direction D3.

The recessed portion 11f includes a bottom surface 11g and an inner surface 11h that connects the bottom surface 11g and the protruding surface 11d to each other. In the case of being viewed from the first direction D1, the inner surface 11h has an annular shape surrounding the bottom surface 11g. The inner surface 11h may include an R-shaped surface that connects the bottom surface 11g and the protruding surface 11d to each other.

In a plan view, the bottom surface 11g curves into concave portion backward as it moves away from the end of the recessed portion 11f in the second direction D2. In a cross-section orthogonal to the second direction D2 (side cross-section), the recessed portion 11f (bottom surface 11g) curves into concave portion backward as it moves away from the end of the recessed portion 11f in the third direction D3.

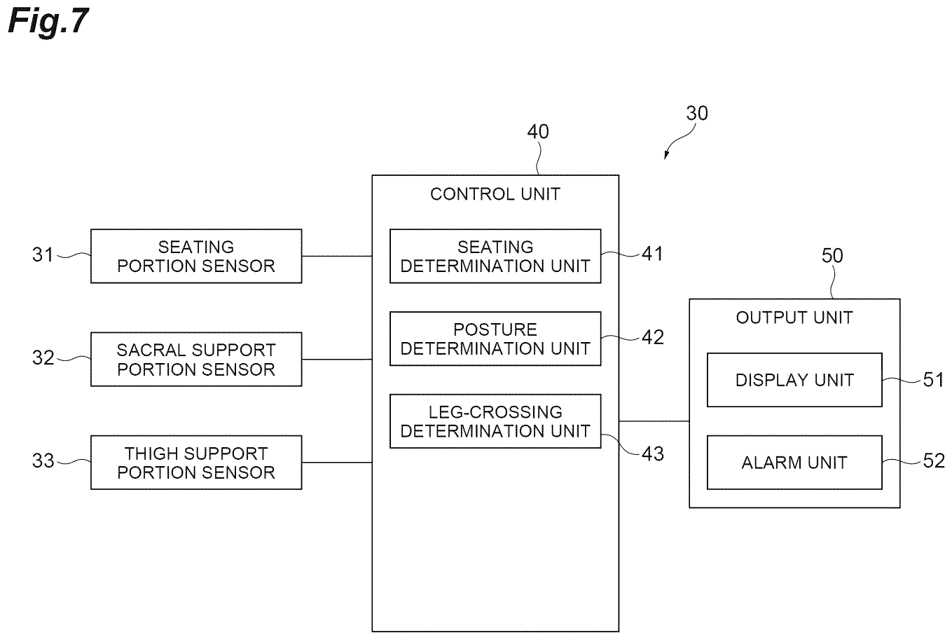

A posture detection system 30 (see FIG. 7) according to the present embodiment detects the posture of a user of the cushion 100 who is seated on the seating portion 2 of the cushion 100. The posture detection system 30 includes a seating portion sensor 31 attached to the seating portion 2, a sacral support portion sensor 32 attached to the sacral support portion 11, and a thigh support portion sensor 33 attached to the thigh support portion 5. In FIGS. 1, 2, 4, and 5, the seating portion sensor 31, the sacral support portion sensor 32, and the thigh support portion sensor 33 are illustrated in a simplified manner.

In one example, the core material 1 has a concave portion formed on the upper surface of the seating portion 2 and a concave portion formed on the upper surface of the thigh support portion 5, and the seating portion sensor 31 is embedded in the concave portion of the seating portion 2, and the thigh support portion sensor 33 is embedded in the concave portion of the thigh support portion 5. For example, the sacral support portion sensor 32 is attached to the recessed portion 11f of the sacral support portion 11.

As described above, in one example, the seating portion sensor 31, the sacral support portion sensor 32, and the thigh support portion sensor 33 are directly attached to the core material 1. However, at least one of the seating portion sensor 31, the sacral support portion sensor 32, and the thigh support portion sensor 33 may be indirectly attached to the core material 1. The following describes an example in which the seating portion sensor 31, the sacral support portion sensor 32, and the thigh support portion sensor 33 are indirectly attached to the core material 1.

FIG. 6 is a cross-sectional view schematic illustrating the cushion 100 that includes the core material 1 and the fabric 20 covering the core material 1. In FIG. 6, the illustration of the core material 1 and other components is simplified. As described above, the cushion 100 is used with the core material 1 housed in the fabric 20. In the example of FIG. 6, the posture detection system 30 of the present embodiment has the seating portion sensor 31, the sacral support portion sensor 32, and the thigh support portion sensor 33.

For example, the fabric 20 is an inner fabric to which the seating portion sensor 31, the sacral support portion sensor 32, and the thigh support portion sensor 33 are attached. In other words, the fabric 20 is a sensor fabric to which the seating portion sensor 31, the sacral support portion sensor 32, and the thigh support portion sensor 33 are fixed. The cushion 100 further includes an outer fabric 22 that covers the fabric 20 functioning as an inner fabric. The fabric 20 functioning as an inner fabric covers, for example, only a part of the core material 1. As a specific example, the fabric 20 is fixed to the core material 1 in such a way as to cover only the seating portion 2 and the sacral support portion 11.

The fabric 20 has the seating portion sensor 31, a sacral support portion sensor 32, and a thigh support portion sensor 33 fixed to it. The seating portion sensor 31, the sacral support portion sensor 32, and the thigh support portion sensor 33 are, for example, piezoelectric sensors fixed to the fabric 20. In this case, the seating portion sensor 31, the sacral support portion sensor 32, and the thigh support portion sensor 33 each measure the pressure exerted on each part of the cushion 100 from the body of the user of the cushion 100, which is placed on the cushion 100.

The type of sensor used for each of the seating portion sensor 31, the sacral support portion sensor 32, and the thigh support portion sensor 33 is not limited to a particular type. For example, at least one of the seating portion sensor 31, the sacral support portion sensor 32, and the thigh support portion sensor 33 may be a strain sensor (or strain gauge) that measures the surface strain of the core material 1.

For example, each of the seating portion sensor 31, the sacral support portion sensor 32, and the thigh support portion sensor 33 may be a thread-like sensor fixed to the fabric 20 through embroidery. In the case where the fabric 20 covers the core material 1, the seating portion sensor 31 is located on the upper surface of the seating portion 2, the sacral support portion sensor 32 is located at the sacral support portion 11, and the thigh support portion sensor 33 is located at the thigh support portion 5.

The outer fabric 22 covers the core material 1 and the fabric 20. The outer fabric 22 covers, for example, the entirety of the core material 1 and the fabric 20. The outer fabric 22 is provided, for example, to prevent the fabric 20 and the core material 1 from getting dirty. The outer fabric 22 may be detachable from the core material 1 and the fabric 20. In one example, the outer fabric 22 may have a fastener (not illustrated), allowing the core material 1 and the fabric 20 to be freely inserted and removed from the inside of the outer fabric 22 in the state where the fastener is opened. In this case, the outer fabric 22 can be removed from the core material 1 and the fabric 20 and washed separately.

As illustrated in FIGS. 1 and 6, in one example, the posture detection system 30 has two seating portion sensors 31 arranged along the second direction D2, one sacral support portion sensor 32, and two thigh support portion sensors 33 arranged along the second direction D2. The seating portion sensors 31 are, for example, arranged around each of the paired ischial support portions 6. In one example, the seating portion sensor 31 is arranged between the ischial support portion 6 and the ventilation hole 2b. Additionally, the seating portion sensor 31 may also be arranged on the buttock support portion 4. In this case, the seating portion sensor 31 measures the contact of the user's buttocks with the buttock support portion 4.

For example, the sacral support portion sensor 32 is fixed to the bottom surface 11g of the recessed portion 11f. However, the sacral support portion sensor 32 may be fixed to the inner surface 11h of the recessed portion 11f, or to the protruding surface 11d of the sacral support portion 11. The sacral support portion sensor 32 may be provided on the inside of the outer edge of the sacral support portion 11 as viewed from the first direction D1. For example, the sacral support portion sensor 32 measures the load from the user's sacrum on the sacral support portion 11.

Each of the paired thigh support portion sensors 33 is fixed to the front portion of the ventilation hole 2b in the thigh support portion 5. The thigh support portion sensors 33 may be arranged on both sides of the convex portion 8 in the second direction D2. The thigh support portion sensor 33 may be provided on the inside of the thigh support portion 5 as viewed from the third direction D3. For example, the thigh support portion sensor 33 measures the load from the user's thigh to the thigh support portion 5.

For example, the seating portion sensor 31 measures whether or not the user of the cushion 100 is seated on the seating portion 2. The seating portion sensor 31 measures, for example, whether or not the user's ischium is in contact with the seating portion sensor 31. The description mentioned above is an example in which the number of seating portion sensors 31 is two. However, the number of the seating portion sensors 31 may be one or three or more and is not limited to a particular count.

For example, the sacral support portion sensor 32 detects the posture of the user of the cushion 100 seated on the seating portion 2. The sacral support portion sensor 32 measures, for example, whether or not the user's sacrum is in contact with the sacral support portion sensor 32 and the load applied by the user's body in contact with the sacral support portion sensor 32. For example, the thigh support portion sensor 33 measures the load applied by the thighs of the user of the cushion 100 seated on the seating portion 2.

The posture detection system 30 includes, for example, a cable 34 and a communication unit 35. The cable 34 is electrically connected to the seating portion sensor 31, the sacral support portion sensor 32, and the thigh support portion sensor 33, and it extends from the fabric 20 to the outside of the fabric 20. The communication unit 35 is provided on the side opposite to the cable 34 relative to the fabric 20.

The communication unit 35 is, for example, a communication board that communicates the measurement data obtained by the seating portion sensor 31, the sacral support portion sensor 32, and the thigh support portion sensor 33. The communication unit 35 receives the measurement data obtained by the seating portion sensor 31, the sacral support portion sensor 32, and the thigh support portion sensor 33 via the cable 34. The cable 34 may be omitted, and the communication unit 35 may be integrated within the fabric 20.

FIG. 7 is a block diagram illustrating the functional configuration of the posture detection system 30. As illustrated in FIG. 7, the posture detection system 30 includes a control unit 40 and an output unit 50. The control unit 40 is, for example, a server. The seating portion sensor 31, the sacral support portion sensor 32, and the thigh support portion sensor 33 are capable of communicating with the control unit 40.

The seating portion sensor 31, the sacral support portion sensor 32, and the thigh support portion sensor 33 each transmit the measurement data obtained by the measurement to the control unit 40 via the communication unit 35. The control unit 40 is provided with a processor (e.g., a CPU), which executes an operating system and application programs, a main memory unit, which includes ROM and RAM, an auxiliary memory unit, which includes a hard disk, flash memory, and a communication control unit, which includes a network card or wireless communication module. However, the control unit 40 is not limited to the above exemplary configuration and can be appropriately modified.

Each functional element of the control unit 40 is implemented by loading specific software into the processor or the main memory unit and executing the software. The processor, in accordance with the software, operates the aforementioned communication control unit and performs data read and write operations on the main memory unit or the auxiliary memory unit. The data or database required for the processing by the control unit 40 is stored in the main memory unit or the auxiliary memory unit.

The control unit 40 may be equipped with a posture detection program. The posture detection program includes, for example, a main module, a data acquisition module, an analysis module, and an output module. The main module is a module that manages the overall functions of the posture detection system 30. The data acquisition module acquires measurement data from each of the seating portion sensor 31, the sacral support portion sensor 32, and the thigh support portion sensor 33. A calculation module performs calculations to evaluate the sitting state and posture of the user of the cushion 100 from the measurement data. The output module outputs the results calculated by the calculation module.

The functional components of the posture detection system 30 (posture detection program) function through the execution of the data acquisition module, the calculation module, and the output module. The posture detection program may be provided, for example, by being recorded on a recording medium such as a CD-ROM, a DVD-ROM, or semiconductor memory. The posture detection program may be provided as a data signal superimposed on a carrier wave via a communication network.

In one example, the posture detection system 30 has a posture data analysis tool. The posture detection system 30 collects, analyzes, and visualizes measurement data for each fixed time period (e.g., one hour or one day) using, for example, a spreadsheet software. For example, the posture detection system 30 visualizes data regarding the user's seating time period, seating frequency, and posture on the cushion 100. In this case, the user can easily grasp the seating time period, seating frequency, and posture on the cushion 100. This result makes it possible to encourage the users to improve their posture.

For example, the control unit 40 includes a seating determination unit 41 and a posture determination unit 42 as functional components. The control unit 40 may also include a leg-crossing determination unit 43 that determines whether or not the legs of user seated on the seating portion 2 are crossed. The seating determination unit 41, the posture determination unit 42, and the leg-crossing determination unit 43 are functions that are implemented, for example, by a program installed in the control unit 40.

The seating determination unit 41 determines whether or not the user is seated on the seating portion 2. The seating determination unit 41 measures the time period during which the user is seated on the seating portion 2. The seating determination unit 41, for example, uses measurement data from the seating portion sensor 31 to determine whether or not the user is seated on the seating portion 2.

The seating determination unit 41, for example, determines that the user is seated on the seating portion 2 if the user's body (e.g., buttocks or ischium) is in contact with the seating portion sensor 31. In the case where a plurality of the seating portion sensors 31 is provided, the seating determination unit 41 may determine that the user is seated on the seating portion 2 if the user's body is in contact with any of the multiple seating portion sensors 31.

The posture determination unit 42 determines the posture of the user seated on the seating portion 2. The posture determination unit 42 determines the posture of the user base on the result measured by the sacral support portion sensor 32. The posture determination unit 42 determines that, for example, the user's posture is improper, such as a slouched posture (hunched back), if the user's body (e.g., sacrum) is not in contact with the sacral support portion sensor 32.

The posture determination unit 42 measures the time period during which the user is hunched. In the case where the user's posture is hunched, the user leans forward, and the sacrum does not come into contact with the sacral support portion sensor 32. If the seating determination unit 41 determines that the user is seated on the seating portion 2 and the sacrum does not come into contact with the sacral support portion sensor 32, the posture determination unit 42 determines that the user is hunched.

The posture determination unit 42 determines that the user's posture is excessively leaning backward if, for example, the load measured by the sacral support portion sensor 32 is equal to or greater than a predetermined value. The posture determination unit 42 measures the time period of excessive backward leaning. In the case where the user is excessively leaning backward, a larger backward load is applied to the sacral support portion sensor 32 compared to a proper posture. In the case where this load is measured by the sacral support portion sensor 32, the posture determination unit 42 determines that the posture is excessively leaning backward and is improper. For example, the sacral support portion sensor 32 may measure the amount of deformation (degree of crushing) of the sacral support portion 11, and the posture determination unit 42 may determine that the user is excessively leaning backward if the measured amount of deformation of the sacral support portion 11 is equal to or greater than a predetermined value.

The posture determination unit 42 determines that the user's posture is proper, if, for example, the load measured by the sacral support portion sensor 32 is less than a predetermined value. The posture determination unit 42 measures the time period during which the posture is proper. In the case where the posture is proper, the back of the user seated on the seating portion 2 extends along the vertical direction, and the sacral support portion 11 receives a moderate load from the sacrum. In the case where this moderate load is measured by the sacral support portion sensor 32, the posture determination unit 42 determines that the posture is proper. Similarly, the posture determination unit 42 may determine that the posture is proper if the measured amount of deformation of the sacral support portion 11 is less than a predetermined value.

The posture determination unit 42 may calculate the difference in the loads on the thigh support portion sensors 33, which are measured by the pair of thigh support portion sensors 33. In this case, the posture determination unit 42 calculates the absolute value of the difference between the load on one of the thigh support portion sensors 33 received from the left thigh and the load on the other of the thigh support portion sensors 33 received from the right thigh.

Then, the posture determination unit 42 may determine that the balance between the left and right sides is unsatisfactory and the posture is improper if the calculated absolute value is equal to or greater than a predetermined value; meanwhile, it may determine that the balance between the left and right sides is satisfactory if the calculated absolute value is not equal to or greater than the predetermined value. In this case, it is possible to grasp whether the posture of the user seated on the seating portion 2 is biased to either the left or right. For example, the posture determination unit 42 measures the time period during which the balance between the left and right sides is unsatisfactory.

The leg-crossing determination unit 43 determines whether or not the legs of the user seated on the seating portion 2 are crossed. The leg-crossing determination unit 43, for example, uses measurement data from the thigh support portion sensor 33 to determine whether or not the user's legs are crossed. The leg-crossing determination unit 43 measures the time period during which the user's legs are crossed. As a specific example, the leg-crossing determination unit 43 determines that the legs are crossed if one of the paired thigh support portion sensors 33 receives a load applied by the thigh and the other thigh support portion sensor 33 does not receive a load applied by the thigh.

Moreover, sitting for an extended period with legs crossed can potentially cause pelvic misalignment, which is undesirable. In the present embodiment, the leg-crossing determination unit 43 determines whether or not the legs of the user seated on the seating portion 2 are crossed, making it possible to notify the user that it is better to avoid crossing the legs for prolonged periods.

The output unit 50 outputs the result of the determination made by each of the seating determination unit 41 and the posture determination unit 42. The output unit 50 may output the result of the determination made by the leg-crossing determination unit 43. The output unit 50 has, for example, a display unit 51 that displays the determination result, and an alarm unit 52 that outputs an alarm in the case where the posture determination unit 42 determines that the posture is improper (e.g., the case where it is determined that the posture is hunched, leaning backward excessively, or biased to the left or right).

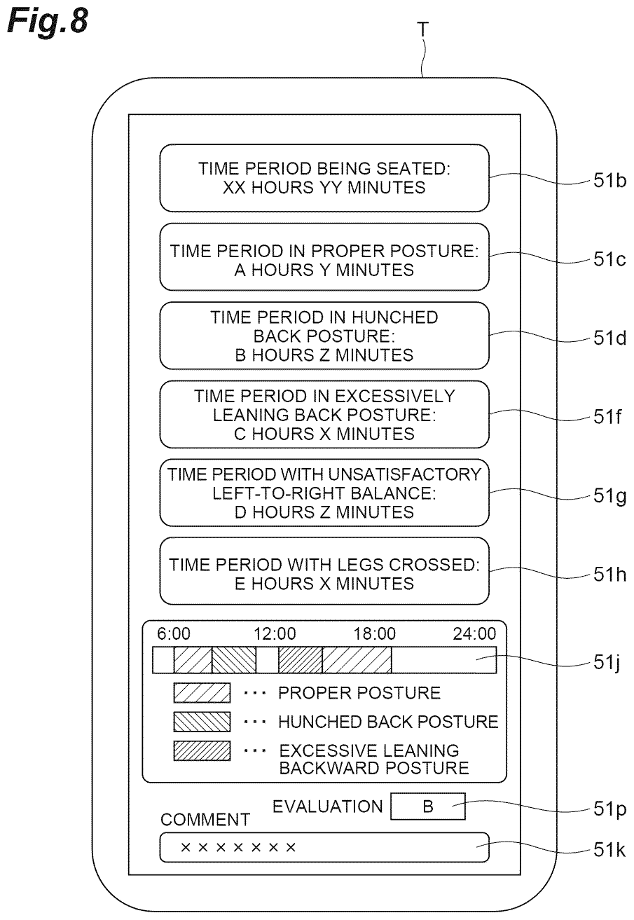

As illustrated in FIGS. 7 and 8, the display unit 51 displays, for example, the determination results made by the seating determination unit 41, the posture determination unit 42, and the leg-crossing determination unit 43 on the display of an information terminal T. The information terminal T is, for example, a mobile terminal. The term “mobile terminal” refers to a portable information terminal such as a mobile phone including smartphones, a tablet, or a notebook computer. The information terminal may be a terminal other than a mobile terminal, and it could also be, for example, a personal computer.

The display unit 51 displays, for example, a seating time period 51b during which the user is seated on the seating portion 2, which is measured by the seating determination unit 41, and a time period 51c during which the posture is proper, which is measured by the posture determination unit 42. In addition, the display unit 51 may also display a time period 51d during which the user is hunched, which is measured by the posture determination unit 42, and a time period 51f during which the user is excessively leaning backward, which is measured by the posture determination unit 42. Furthermore, the display unit 51 may also display a time period 51g during which a left-to-right balance is unsatisfactory, which is measured by the posture determination unit 42, and a time period 51h during which the legs are crossed, which is measured by the leg-crossing determination unit 43.

The display unit 51 may display, as a time chart 51j, at least one of the seating time period during which the user is seated on the seating portion 2, the time period during which the posture is proper, the time period during which the posture is hunched, the time period during which the posture is excessively leaning backward, the time period during which a left-to-right balance is unsatisfactory, and the time period during which the user's legs are crossed. In this case, it is possible to grasp each of the above-mentioned time periods at a glance by simply viewing the time chart 51j. Thus, the user is able to grasp the sitting habits on the cushion 100, enabling the avoidance of issues such as leg pain by correcting unsatisfactory sitting posture.

The display unit 51 may display, as a comment 51k, the determination result obtained by the seating determination unit 41 and the determination result obtained by the posture determination unit 42. Additionally, the display unit 51 may also display the determination result obtained by the leg-crossing determination unit 43 as the comment 51k. Furthermore, the display unit 51 may also display, as a ranking 51p (evaluation), the determination result obtained by the seating determination unit 41 and the determination result obtained by the posture determination unit 42.

Examples of the comment 51k may include statements such as “You spend excessive time sitting, so you should stand up and exercise a little”, “You often sit with a hunched back, so be conscious of sitting with your back straight”, “You spend a lot of time leaning back, so put your stomach in and straighten your back”, “Be conscious of your left and right balance while sitting”, “Try to shorten the time you cross your legs”, and “You are sitting in the right posture”. By looking at the comment 51k, the user is able to grasp how to sit on the cushion 100 in a way that promotes good health.

The alarm unit 52 outputs an alarm if the posture determination unit 42 determines that the posture is improper. The alarm unit 52 notifies the user's information terminal T of an alarm, for example, if the posture determination unit 42 determines that the posture is improper. The alarm may be notified via e-mail or may be notified through an application.

The alarm unit 52 outputs an alarm if, for example, any of the following time periods is equal to or greater than a predetermined period of time: the seating time period during which the user is seated on the seating portion 2, the time period during which the posture is hunched, the time period during which the posture is excessively leaning back, the time period during which a left-to-right balance is unsatisfactory, and the time period during which the user's legs are crossed. In one example, the alarm unit 52 may be a lamp attached to the cushion 100. In this case, the user can easily grasp the state of sitting too long or having an unsatisfactory posture by looking at the lamp on the cushion 100 on which the user is sitting. The alarm unit 52 may also output a sound if any of the following time periods is equal to or greater than a predetermined period of time: the seating time period during which the user is seated on the seating portion 2, the time period during which the posture is hunched, the time period during which the posture is excessively leaning back, the time period during which a left-to-right balance is unsatisfactory, and the time period during which the user's legs are crossed.

Subsequently, a specific exemplary posture detection method according to the present embodiment is described. In one example, a method of detecting the posture of a user seated on the cushion 100 illustrated in FIG. 6 is described. For example, the fabric 20 is fixed to the core material 1 in such a manner that the positions of the seating portion sensor 31, the sacral support portion sensor 32, and the thigh support portion sensor 33 are aligned with the positions of the buttock support portion 4, the sacral support portion 11, and the thigh support portion 5, respectively. Then, the core material 1 and the fabric 20 are housed inside the outer fabric 22.

The seating portion sensor 31, attached to the seating portion 2, measures whether or not the user is seated on the seating portion 2 (step of measurement by the seating portion sensor). For example, the seating portion sensor 31 measures whether or not the buttocks are in contact with the seating portion sensor 31, and the seating determination unit 41 determines whether or not the user is seated on the seating portion 2.

In addition, the sacral support portion sensor 32 measures the posture of the user who is seated on the seating portion 2 (step of measurement by the sacral support portion sensor). The sacral support portion sensor 32 measures whether the sacrum is in contact with the sacral support portion sensor 32 and measures the load from the sacrum to the sacral support portion sensor 32. For example, the posture determination unit 42 determines that the user has a hunched back if the sacrum is not in contact with the sacral support portion sensor 32, determines that the user is excessively leaning backward if the load on the sacral support portion sensor 32 is equal to or greater than a predetermined value, and determines that the posture is proper if the load on the sacral support portion sensor 32 is not equal to or greater than the predetermined value (step of determination). The paired thigh support portion sensors 33 may measure the load applied by the thighs, and the posture determination unit 42 may determine whether or not the balance between the left and right is proper. The leg-crossing determination unit 43 may determine whether or not the user's legs are crossed using the measurement results obtained by the pair of thigh support portion sensors 33.

If the posture determination unit 42 determines that the user's posture is satisfactory, then, for example, the display unit 51 displays that the user's posture is correct, and the series of steps is completed. If the posture determination unit 42 determines that the user's posture is unsatisfactory, the alarm unit 52 outputs an alarm (step of outputting an alarm). Specifically, the alarm unit 52 may notify the information terminal T that the posture is improper, or a lamp on the cushion 100 may be made to light up. The alarm unit 52 may notify the user that the posture is improper through vibration. In this case, the alarm unit 52, for example, causes the cushion 100 on which the user is seated to vibrate. After the alarm unit 52 has finished the notification, the series of steps of the posture detection method is completed.

The operational effects and benefits obtained from the posture detection system 30 and the posture detection method according to the present embodiment are now described in more detail. In the posture detection system 30 and the posture detection method according to the present embodiment, the cushion 100 includes the seating portion 2 having the buttock support portion 4 that supports the buttocks of the user, and also includes the sacral support portion 11 that supports the sacrum of the user from behind. The posture detection system 30 includes a seating portion sensor 31 that measures whether or not the user is seated on the seating portion 2 of the cushion 100. The measurement of whether or not the user is seated on the seating portion 2 by the seating portion sensor 31 allows the time period during which the user is seated on the seating portion 2 to be calculated.

The user can grasp whether or not a long period of sitting has occurred, thereby avoiding issues such as impaired circulation in the legs and lower back or a decline in muscle strength. The posture detection system 30 includes the sacral support portion sensor 32 provided on the sacral support portion 11 and configured to measure the posture of the user seated on the cushion 100. The sacrum is apart that protrudes from the user's back. Providing the sacral support portion sensor 32 on the sacral support portion 11 that supports the sacrum allows the user's posture to be measured with high accuracy.

The posture detection system 30 includes the alarm unit 52 that outputs an alarm if the posture determination unit 42 determines that the posture is unsatisfactory. Outputting an alarm by the alarm unit 52 if the posture is unsatisfactory enables the user to grasp that the user's posture is poor. As a result, the possibility of physical strain caused by poor posture can be reduced.

In the present embodiment, the sacral support portion sensor 32 measures whether or not the user's sacrum is in contact with the sacral support portion sensor 32. The posture determination unit 42 determines that the user's posture is hunched if the user's sacrum is not in contact with the sacral support portion sensor 32. In the case where the user leans forward and the sacrum is not in contact with the sacral support portion sensor 32, the posture determination unit 42 determines that the user's posture is hunched. The determination of whether or not the user is hunched based on the presence or absence of contact with the sacral support portion sensor 32 enables the user's posture to be grasped with high accuracy and hunched back to be mitigated.

In the present embodiment, the sacral support portion sensor 32 measures the load applied by the user's sacrum on the sacral support portion sensor 32. The posture determination unit 42 determines that the user is excessively leaning backward if the load measured by the sacral support portion sensor 32 is equal to or greater than a predetermined value. In the case where the load on the sacral support portion sensor 32 is equal to or greater than a predetermined value, the posture determination unit 42 determines that there is excessive leaning backward. The determination of whether or not the user is excessively leaning backward using the load on the sacral support portion sensor 32 enables the suppression of excessive backward leaning and a decline in abdominal muscle strength.

In the present embodiment, the seating portion 2 has the pair of thigh support portions 5 aligned along the left-to-right direction (second direction D2) as viewed from the user seated on the seating portion 2. The posture detection system 30 further includes the pair of thigh support portion sensors 33, each attached to one of the paired thigh support portions 5, which measure the load on the thighs of the user seated on the seating portion 2. The measurement of the load on the user's left and right thighs by the pair of thigh support portion sensors 33, respectively, makes it possible to grasp the balance between the left and right sides of the thighs. In the case where one of the paired thigh support portion sensors 33 detects the load and the other of the paired thigh support portion sensors 33 does not detect the load, it can be determined that the user's legs are likely to be crossed. Thus, the posture of the user being seated on the cushion 100 can be grasped with a higher degree of accuracy.

In the present embodiment, the posture detection system 30 includes the fabric 20, to which the seating portion sensor 31 and the sacral support portion sensor 32 are attached and which covers at least a part of the core material 1 of the cushion 100. In this case, the seating portion sensor 31 and the sacral support portion sensor 32 are attached to the fabric 20, and at least a part of the core material 1 is covered by the fabric 20. Covering the core material 1 of an existing cushion 100 with the fabric 20 enables the seating portion sensor 31 and the sacral support portion sensor 32 to be attached to the cushion 100. Thus, it is possible to easily arrange the seating portion sensor 31 and the sacral support portion sensor 32 on the existing cushion 100.

In the present embodiment, the posture detection system 30 includes the outer fabric 22 that covers the core material 1, and the fabric 20 is the inner fabric that is covered by the outer fabric 22. In this case, the outer fabric 22 is provided on the outside of the inner fabric (fabric 20) to which the seating portion sensor 31 and the sacral support portion sensor 32 are attached. The outer fabric 22 can be removed and washed, so the cushion 100 can be maintained in a more hygienic condition, allowing for prolonged use of the cushion 100.

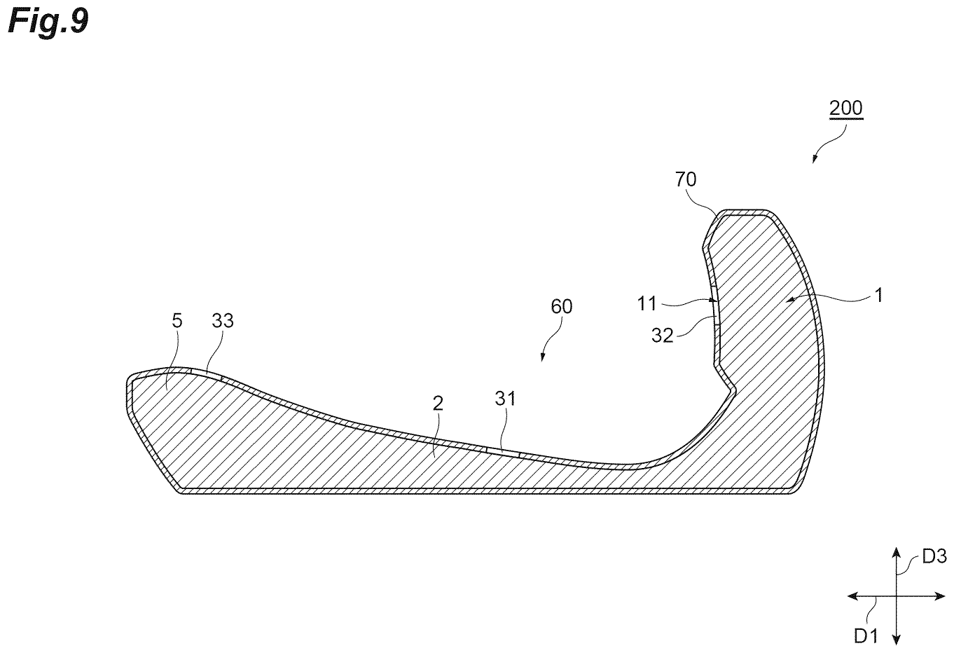

The following describes a cushion 200 equipped with a posture detection system 60 according to a modification with reference to FIG. 9. The cushion 200 differs from the cushion 100 described above in that it does not include an inner fabric. Some components of the posture detection system 60 and the cushion 200 are identical to those of the posture detection system 30 and the cushion 100. In the following, descriptions that overlap with the posture detection system 30 and the cushion 100 will be omitted as appropriate by assigning the same reference numerals.

The cushion 200 is used with the core material 1 housed in a fabric 70. The fabric 70 is provided to prevent the core material 1 from getting dirty. The fabric 70 is an outer fabric to which the seating portion sensor 31, the sacral support portion sensor 32, and the thigh support portion sensor 33 are attached. The fabric 70 as the outer fabric, for example, covers the entirety of the core material 1.

The fabric 70 may be detachable from the core material 1. For example, the fabric 70 may have a fastener (not illustrated), allowing the core material 1 to be easily inserted and removed from the inside of the fabric 70 in the state where the fastener is opened. In this case, the seating portion sensor 31, the sacral support portion sensor 32, and the thigh support portion sensor 33 can be attached to or detached from the core material 1.

In the present modification, the posture detection system 60 includes the fabric 70 to which at least the seating portion sensor 31 and the sacral support portion sensor 32 are attached and which covers at least a part of the core material 1 of the cushion 200. The seating portion sensor 31 and the sacral support portion sensor 32 are attached to the fabric 70, and the core material 1 is covered by the fabric 70. The seating portion sensor 31 and the sacral support portion sensor 32 can be attached to the cushion 200 by covering the core material 1 of the existing cushion 200 with the fabric 70, so enabling the seating portion sensor 31 and the sacral support portion sensor 32 to be easily arranged on the existing cushion 200. The present modification achieves similar operational effects to those in the embodiment described above.

The description above is given of the embodiments of the posture detection system and the posture detection method according to the present disclosure. The posture detection system and the posture detection method according to the present disclosure are not limited to the embodiments described above, but modifications and applications to other implementations can be made without departing from the spirit and scope of the invention as set forth in the claims. The configuration and function of each component of the posture detection system, as well as the content and sequence of steps in the posture detection method, can be appropriately altered as long as the above sprit and scope of the invention remains unchanged. The shape, size, number, material, and arrangement of each part of the cushion are not limited to the cushion 100 or 200 described above, and can be appropriately modified.

For example, in the embodiment described above, the described example involved the thigh support portion sensor 33 measuring the load applied by the thigh. However, the thigh support portion sensor 33 may be omitted. In the embodiment described above, the core material 1 including the seating portion 2 and the lumbar support portion 3 has been described. For example, the height of the lumbar support portion 3 can be adjusted as appropriate. For example, the height of the upper end of the lumbar support portion 3 may be the height of the user's back, the height of the user's shoulders, or the height of the user's head, and is not particularly limited. In this case, the cushion can be used as a chair such as a gaming chair.

For example, in the above embodiment, the example described involved the alarm unit 52 notifying the information terminal T that the posture is improper. However, for example, the alarm unit may output an alarm as sound. The alarm unit may also output an alarm as light. The alarm unit may also output an alarm as vibration. In this way, the type of alarm output by the alarm unit can be modified as appropriate.

For example, the lumbar support portion may be a cushion that is foldable relative to the seating portion. In this case, the cushion can be used as an office chair, a reclining chair, or a fully flat seat. The cushion may also be a seat used for transportation machinery of vehicles such as automobiles, railway cars, or aircraft. In this manner, the posture detection system and the posture detection method according to the present disclosure are applicable to various cushions.

The following describes a modification of a cushion to which the posture detection system and the posture detection method can be applied. In the present modification, a lumbar support is provided in addition to a body of cushion. The lumbar support and the cushion are constituted by a flexible material. The term “flexible material” refers to a cushioning material that deforms in response to the load applied by a person's body placed on it. In one example, the lumbar support and the cushion are placed on a chair. The term “chair” is something on which the user of the lumbar support and the cushion to sits.

The term “lumbar support” refers to a component used to support the user's lower back (lumbar spine). For example, the lumbar support fits between the backrest of the chair and the user's lower back, so that the lower back of the user seated on the chair is supported by the lumbar support. The term “user” refers to users of the lumbar support or cushion, and, for example, refers to a person who places the lumbar support behind their lower back or someone seated on the cushion.

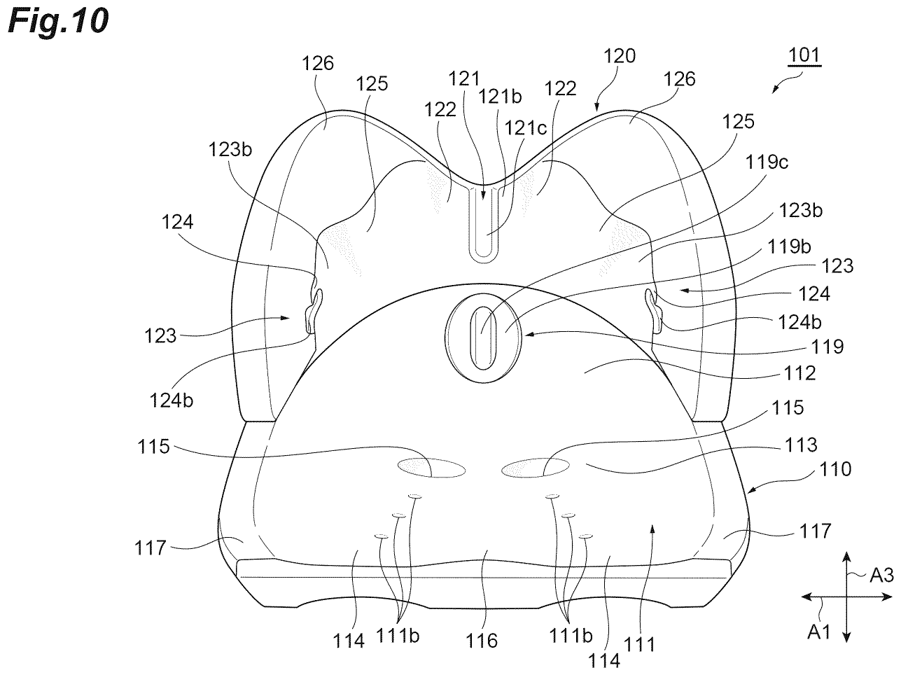

FIG. 10 is a front view illustrating a lumbar support 120 and a cushion 101. The cushion 101 includes the lumbar support 120 and a cushion portion 110, which is located below the lumbar support 120 and has a seating portion 111 for the user to sit on.

The cushion portion 110 is now described. As illustrated in FIG. 10, the cushion portion 110 includes the seating portion 111 that extends horizontally and a lumbar support portion 112 that extends upward from the seating portion 111. The cushion portion 110 is constituted by a flexible material. For example, the cushion portion 110 is constituted by polyurethane foam.

In one example, the cushion portion 110 is formed by urethane molding. In the case where the cushion portion 110 is formed by urethane molding constituted by polyurethane foam, the cushion portion 110 constituted by a flexible material can be easily manufactured. The cushion portion 110 is used, for example, by being housed in a covering fabric.

Moreover, the flexible material mentioned above may include a thermoplastic elastomer. The flexible material may be a material with elasticity sufficient to deform in response to the load applied by a person's body placed on it. The flexible material concerned may be a resin (e.g., polypropylene, polyester, or polyethylene) that deforms in response to the load applied by a person's body placed on it. The type of flexible material may be modified as appropriate.

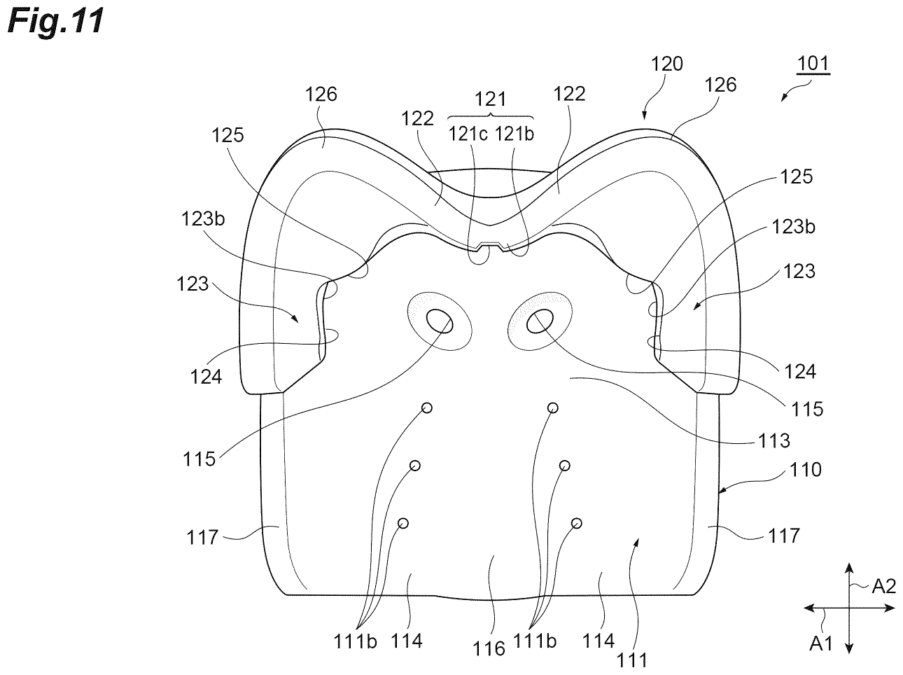

FIG. 11 is a plan view illustrating the cushion 101. As illustrated in FIGS. 10 and 11, the seating portion 111 extends in a first direction A1 and a second direction A2 that intersects with the first direction A1. The first direction A1 is the left-to-right direction as viewed from the user seated on the seating portion 111, while the second direction A2 is the front-to-rear direction as viewed from the user seated on the seating portion 111. The seating portion 111 has a thickness in a third direction A3 that intersects with both the first direction A1 and the second direction A2. For example, the third direction 83 is a vertical direction.

In the following description, the forward direction as viewed from the user seated on the seating portion 111 may be referred to as “front”, “front side”, or “forward”, while the opposite direction to the forward direction may be referred to as “rear”, “rear side”, or “backward”. However, these directions are for convenience of description and are not intended to limit the position, orientation, or the like of each component.

The seating portion 111 has a buttock support portion 113 on which the user's buttocks are placed, and a thigh support portion 114 on which the user's thighs are placed. The buttock support portion 113 and the thigh support portion 114 are arranged to be aligned along the second direction A2. The buttock support portion 113 has a pair of ischial support portions 115 that support the ischium of the user seated on the seating portion 111.

For example, the seating portion 111 has a ventilation hole 111b. The ventilation hole 111b is formed, for example, on the upper surface of the buttock support portion 113 and the upper surface of the thigh support portion 114. The ventilation hole 111b penetrates, for example, the seating portion 111 in the third direction A3. The upper end of the inner wall defining the ventilation hole 111b may be R-chamfered to be rounded. In a plan view, the multiple ventilation holes 111b are arranged to align from the rear to the front.

For example, in a plan view (a plan view in which the lumbar support portion 112 is oriented to be located above the seating portion 111), a plurality (e.g., six) of ventilation holes 111b may be arranged on both sides (left and right sides) in the first direction A1. For example, three ventilation holes 111b are formed on each side in the first direction A1.

For example, two ventilation holes 111b are aligned along the first direction A1, and the distance between the two ventilation holes 111b aligned along the first direction A1 increases from the buttock support portion 113 toward the thigh support portion 114 (in the forward direction). In one example, the multiple ventilation holes 111b are arranged in such a manner that they form a V-shape in a plan view.

For example, in the cushion portion 110, the ischial support portion 115 is formed by urethane molding, and the ventilation hole 111b is formed by punching. In this case, the part of the mold used to manufacture the cushion portion 110 that forms the ventilation hole 111b can be eliminated to be needed, so the shape of the mold can be simplified. In molding liquid urethane using the mold, an escape route for air in the liquid urethane can be secured, ensuring a clean finish for the cushion portion 110.

The ischial support portion 115 is formed on the upper surface of the buttock support portion 113 and is indented in the buttock support portion 113. The buttock support portion 113 has the paired ischial support portions 115 aligned along the first direction A1. The ischial support portion 115 has a concave portion that is indented from the upper surface of the buttock support portion 113. The ischial support portion 115 has the shape formed to have a major axis and a minor axis in a plan view. For example, the shape of the ischial support portions 115 is oval (such as an ellipse) in a plan view. However, the shape of the ischial support portions 115 in a plan view is not limited to an oval or ellipse shape and also may be rectangular, rounded-rectangular, rhombic, or rounded-rhombic, and it can be appropriately modified as needed.

The extension line of the major axis of one of the paired ischial support portions 115 intersects with the extension line of the major axis of the other of the paired ischial support portions 115 at an intersection point located closer to the thigh support portion 114 (closer to the front) than the paired ischial support portions 115. The result allows the pair of ischial support portions 115 to form an inverted V shape in a plan view (plan view in the case where the lumbar support portion 112 is oriented to be located above the seating portion 111).

The ischial support portion 115 may have a through-hole penetrating the buttock support portion 113 in the third direction A3. In this case, the ischial support portion 115 has an inner wall that defines the through-hole and a tapered surface located between and connecting the inner wall and the upper surface of the buttock support portion 113. For example, the area of the ischial support portion 115 as viewed from above is larger than the area of the ischial support portion 115 as viewed from below.

The seating portion 111 may have an inclined surface that extends obliquely from the buttock support portion 113 toward the thigh support portion 114. The inclined surface extends obliquely upward from the buttock support portion 113 toward the thigh support portion 114. The inclined surface is inclined relative to the second direction A2 so that the seating portion 111 becomes thicker as it progresses from the buttock support portion 113 to the thigh support portion 114. The seating portion 111 has the paired thigh support portions 114 aligned along the first direction A1 and a convex portion 116 located between the pair of thigh support portions 114. The convex portion 116 indicates a portion that protrudes upward between the pair of thigh support portions 114.

The thigh support portion 114 provides a surface for the undersides of the user's thighs to rest on. The thigh support portion 114 supports the undersides of the user's thighs. For example, the thigh support portion 114 has a concave portion that is indented in the thickness direction (third direction A3) of the cushion portion 110. The concave portion of the thigh support portion 114 is formed, for example, on each of the upper and lower surfaces of the cushion portion 110. For example, the concave portion formed on the lower surface of the cushion portion 110 in the thigh support portion 114 defines a space between a mounting surface, which is the surface on which the cushion 101 is placed, and the concave portion. This space allows the thigh support portion 114 to sink more easily as the thigh is placed on the thigh support portion 114.

The lumbar support portion 112 extends upward from the end (rear end) of the seating portion 111 in the second direction A2. The lumbar support portion 112 has, for example, a sacral support portion 119 located in the center in the first direction A1. The sacral support portion 119 has a convex shape that protrudes forward. In other words, the sacral support portion 119 protrudes forward at the lumbar support portion 112. In one example, the shape of the sacral support portion 119, as viewed from the front, has a major axis and a minor axis. For example, the shape of the sacral support portion 119, as viewed from the front, is an oval shape (such as an ellipse shape).

For example, the sacral support portion 119 has a protruding surface 119b with a forward-protruding curved shape. The height of the protruding surface 119b, measured from the front surface of the lumbar support portion 112, increases as it moves from the outer edge of the protruding surface 119b toward the inside of the protruding surface 119b. For example, the sacral support portion 119 may have a recessed portion 119c indented backward from the protruding surface 119b. The recessed portion 119c is a portion into which the sacrum of the user of the cushion 101 fits.

The recessed portion 119c extends in the first direction A1 and the third direction A3. For example, the length of the recessed portion 119c in the third direction A3 is longer than the length (or width) of the recessed portion 119c in the first direction A1. The shape of the recessed portion 119c as viewed from the second direction A2 may be a groove shape (or an oval shape) extending in the third direction A3.

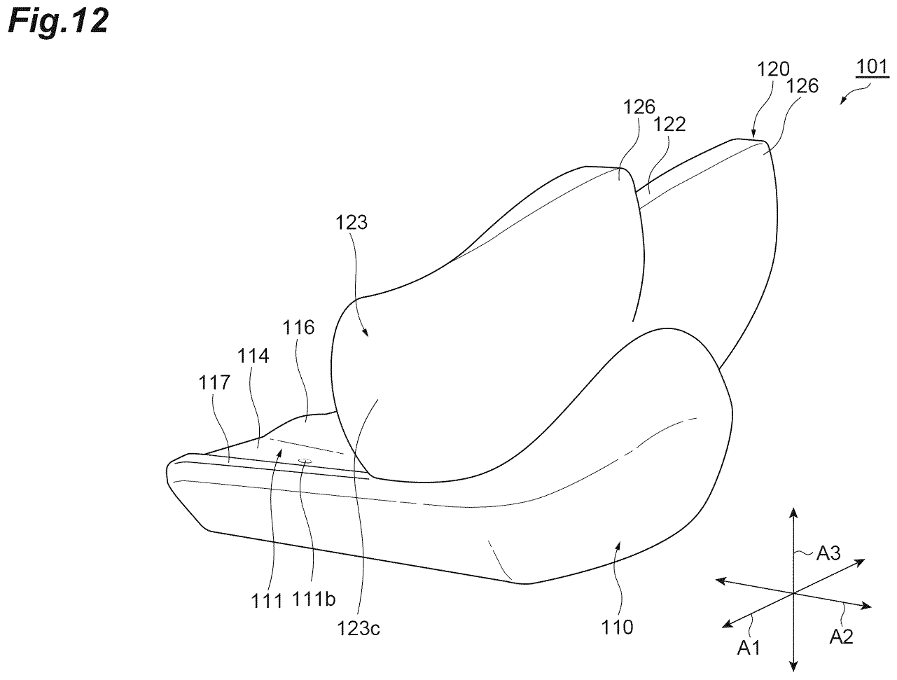

The seating portion 111 has a pair of convex portions 117 located at both ends in the first direction A1 as viewed from the thigh support portion 114. The convex portion 117 extends from each of the paired thigh support portions 114 to the lumbar support portion 112. As illustrated in FIGS. 10 and 12, the convex portion 117 extends from both ends of the thigh support portion 114 in the first direction A1 toward the lumbar support portion 112.

The convex portion 117 is curved to rise upward toward the center in the first direction A1 at the lumbar support portion 112. In the case of being viewed from the second direction A2, the height of the convex portion 117 relative to the thigh support portion 114 increases as it approaches the center of the first direction A1. For example, as viewed from the second direction A2, the convex portion 117 is a mountain-like shape (or inverted U-shape).

In the cushion 101, the lumbar support 120 is placed on the convex portion 117 of the cushion portion 110. The lower surface of the lumbar support 120 is curved to be indented upward as it approaches the center in the first direction A1. In the case of being viewed from the second direction A2, the height of the lower surface of the lumbar support 120 increases as it approaches the center in the first direction A1. For example, as viewed from the second direction A2, the lower surface of the lumbar support 120 has a mountain-like shape (or an inverted U-shape).

The shape of the lower surface of the lumbar support 120 is modeled after the upper surface of the convex portion 117. Contacting the lower surface of the lumbar support 120 with the upper surface of the convex portion 117 of the cushion portion 110 enables the lumbar support 120 to be easily placed on the cushion portion 110. It is also possible to easily remove the lumbar support 120 from the cushion portion 110. The lumbar support 120 may be used together with the cushion portion 110, or may be used alone. The lumbar support 120 may be fastened to the backrest of a chair or may be used in that fastened state.

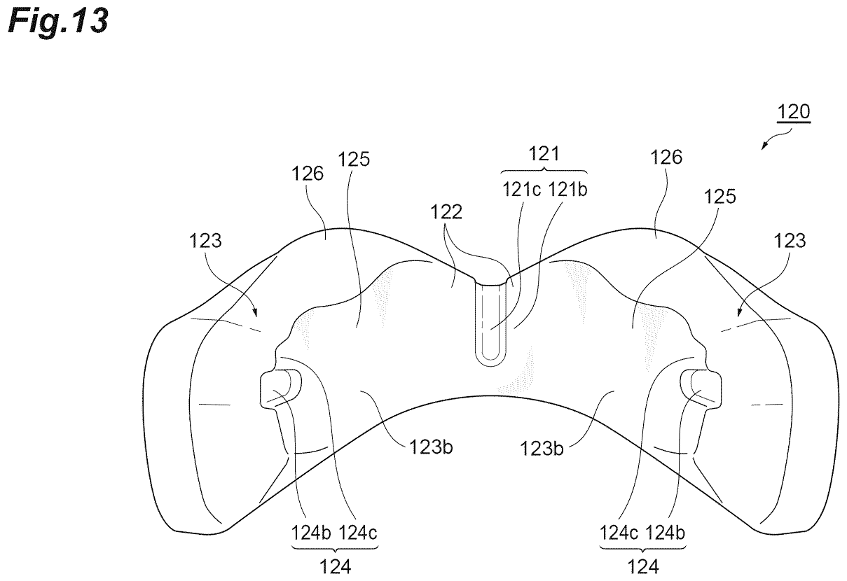

The lumbar support 120 is now described with reference to FIGS. 11, 12, and 13. FIG. 13 is a front view illustrating the lumbar support 120. The lumbar support 120 has a lumbar spine support portion 121 that supports the lumbar spine of the user from behind, and a pair of side portions 123 located on both the left and right sides of the user as viewed from the user whose lumbar spine is supported by the lumbar spine support portion 121. The lumbar support 120 has a pair of connection portions 122 that extend from the lumbar spine support portion 121 to each of the paired side portions 123.

For example, the lumbar support 120 has an M-shape in a plan view (as viewed from the third direction A3). In this case, the part that protrudes downward at the center of the M-shape in a plan view is the lumbar spine support portion 121, and the parts that extend up and down in a plan view at both ends of the M-shape are the side portions 123. The parts that extend from the upper ends of the side portions 123 of the M-shape in a plan view to the lumbar spine support portion 121 are the connection portion 122.

The lumbar support 120 is constituted by a flexible material. For example, the material of the lumbar support 120 is the same as the material of the cushion portion 110. In this case, it is possible to manufacture the lumbar support 120 even more easily. For example, the lumbar support 120 is formed by urethane molding constituted by polyurethane foam. The lumbar support 120 is used, for example, by being housed in a covering fabric. Moreover, the lumbar support 120 may be used alone housed in the covering fabric or may be used together with the cushion portion 110 housed in the covering fabric.

The lumbar spine support portion 121 is formed in a portion that includes the center of the first direction A1 in the lumbar support 120. The lumbar spine support portion 121 has a convex shape that protrudes forward. The lumbar spine support portion 121 protrudes forward from the front surface (the surface facing the front) of the lumbar support 120. For example, the lumbar spine support portion 121 has a protrusion 121b that protrudes toward the back of the user, and a first concave portion 121c that is indented from the protrusion 121b and extends along the third direction A3, which is the height direction of the user's body.

For example, the protrusion 121b has a curved shape that protrudes forward. The protrusion height of the protrusion 121b at the front surface of the lumbar support 120 increases as it approaches the center of the lumbar support 120 in the first direction A1. The first concave portion 121c is a portion that is indented backward from the protrusion 121b. The lumbar spine support portion 121 is located directly above the sacral support portion 119 in the state where the lumbar support 120 is placed on the cushion portion 110.

The first concave portion 121c is a portion into which the lumbar spine of the user of the lumbar support 120 fits. Visual recognition of the first concave portion 121c allows the user to easily grasp that the lumbar support 120 should be attached so that the lumbar spine fits into the first concave portion 121c. The first concave portion 121c can be used as a marker for the lumbar spine support portion 121. This similarly applies to the recessed portion 119c of the sacral support portion 119.

In a plan view (as viewing the lumbar support 120 along the third direction A3 in such a direction that the lumbar support 120 appears M-shaped), the connection portion 122 extends obliquely upward from the lumbar spine support portion 121. Each of the paired connection portions 122 extends obliquely backward from the lumbar spine support portion 121 as viewed by a user whose lumbar spine is supported by the lumbar spine support portion 121.

The connection portion 122 has a plate-like shape in a plan view. For example, the thickness of the connection portion 122 in a plan view is thinner than the thickness of the side portion 123 in a plan view. In one example, the thickness of the connection portion 122 in a plan view is thinner than the thickness of the portion other than the connection portion 122 in a plan view. The lumbar spine support portion 121 and the connection portion 122 can be easily deformed.

In the present embodiment, the lumbar support 120 has a pair of top portions 126 located at the rear end of the respective connection portions 122. The top portions 126 are located at the ends opposite the lumbar spine support portion 121 as viewed from the connection portion 122. In the case where the lumbar support 120 is attached to a user, the height of the connection portion 122 increases as it moves away from the lumbar spine support portion 121, with the top portions 126 reaching the highest point. In a plan view, the pair of top portions 126 correspond to a pair of protrusions located at the upper ends of the M-shape of the lumbar support 120.

The side portion 123 is a portion that extends forward from the top portion 126. Each of the paired side portions 123 has an inner surface 123b that faces the user. Each of the side portions 123 includes an ilium-facing portion 124 that faces the user's ilium. In a plan view, at the end of the ilium-facing portion 124 opposite the top portion 126, there is formed an inclined surface that extends obliquely forward from the ilium-facing portion 124 and an end surface that is located opposite the ilium-facing portion 124 as viewed from the inclined surface.

For example, the ilium-facing portion 124 has an ilium support portion 124c that protrudes from the inner surface 123b and supports the user's ilium, and it has a second concave portion 124b that is indented from the ilium support portion 124c. For example, the ilium support portion 24c has a curved surface that protrudes toward the inside of the lumbar support 120 in a plan view. For example, the second concave portion 124b is a hole formed in the ilium support portion 124c. In one example, the second concave portion 124b extends in a direction inclined with respect to both the first direction A1 and the second direction A2.

The second concave portion 124b is a portion into which at least a part of the user's ilium fits. Visual recognition of the second concave portion 124b allows the user of the lumbar support 120 to easily grasp that the lumbar support 120 should be attached so that the ilium fits into the second concave portion 124b. Similar to the first concave portion 121c and the like described above, the second concave portion 124b can be used as a marker for the ilium support portion 124c.

Each of the paired side portions 123 has a rib support portion 125 that protrudes from the inner surface 123b at a position between the lumbar spine support portion 121 and each of the paired ilium support portions 124c. The rib support portion 125 is a portion that supports the lower part of the ribs of the user of the lumbar support 120. The rib support portion 125 protrudes in a curved shape from the inner surface 123b. In a plan view, the rib support portion 125 protrudes, for example, in an are shape from the inner surface 123b. For example, the lower ends of the user's ribs are supported by the pair of rib support portions 125.

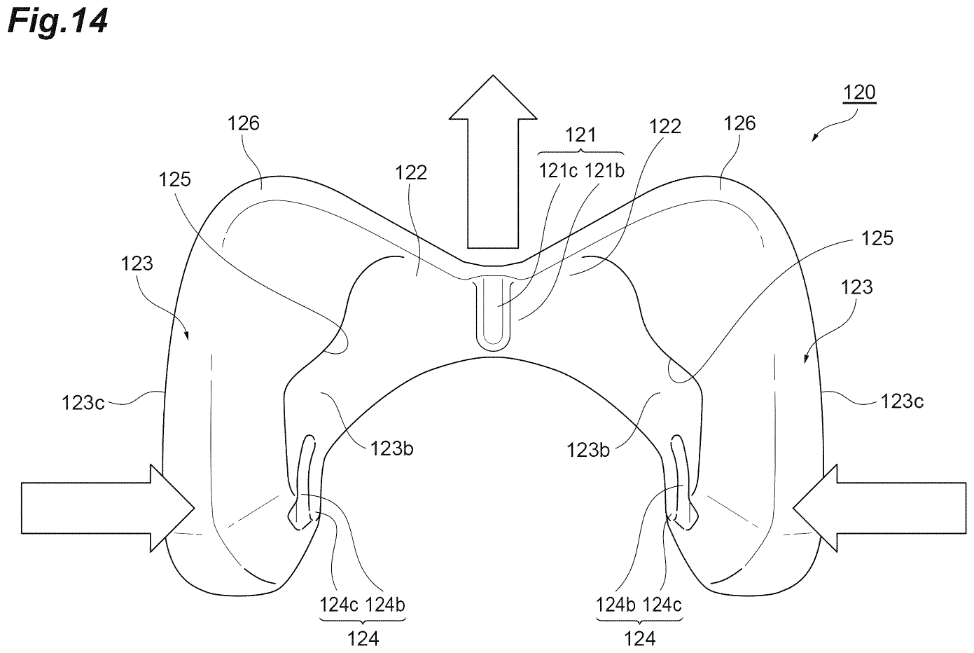

The operational effects obtained from the cushion 101 are now described. As illustrated in FIG. 14, the lumbar support 120 has the lumbar spine support portion 121 that supports the lumbar spine of the user from behind and the pair of side portions 123 located on both the left and right sides of the user, with each side portion 123 and the lumbar spine support portion 121 being connected to each other via the connection portion 122. The lumbar support 120 is constituted by a flexible material, and each of the paired connection portions 122 extends obliquely backward from the lumbar spine support portion 121 and has a plate-like shape in a plan view. In the case where the user's lumbar spine comes into contact with the lumbar spine support portion 121, the pair of connection portions 122 bend backward, and the pair of side portions 123 move toward each other, approaching both sides of the user's waist. While the lumbar spine support portion 121 supports the user's back and lumbar spine, the pair of side portions 123 can support both the left and right sides of the user's waist. By firmly supporting the user's back and both the left and right sides of the waist, it is possible to make the user's posture satisfactory.

Each of the paired side portions 123 may have the inner surface 123b facing the user, and the ilium support portion 124c protruding from the inner surface 123b and supporting the user's ilium. In this case, the user's ilium comes into contact with each of the paired ilium support portions 124c, and each of the ilium support portions 124c supports the user's left and right ilium. By supporting each of the user's ilium, it is possible for the user's waist to be supported more firmly.

The lumbar spine support portion 121 may have the protrusion 121b that protrudes toward the back of the user, and the first concave portion 121c that is indented from the protrusion 121b and extends along the height direction of the user's body. In this case, the user's lumbar spine can be gently supported as it fits into the first concave portion 121c. Visual recognition of the first concave portion 121c of the lumbar spine support portion 121 allows the user to grasp that the lumbar spine should be supported by the lumbar spine support portion 121. The first concave portion 121c can be used as a marker for the lumbar spine support portion 21.

Each of the paired side portions 123 may have a rib support portion 125 that protrudes from the inner surface 123b at a position between the lumbar spine support portion 121 and each of the paired ilium support portions 124c and supports the lower part of the user's ribs. In this case, the lower part of the user's ribs can be supported by the pair of rib support portions 125 located between the lumbar spine support portion 121 and each of the paired ilium support portions 124c.

In the lumbar support 120, each of the paired side portions 123 has the inner surface 123b facing the user, the ilium support portion 124c protruding from the inner surface 123b and supporting the user's ilium, and the second concave portion 124b indented from the ilium support portion 124c. The user's ilium can be gently supported as it fits into the second concave portion 124b. Visual recognition of the second concave portion 124b of the ilium support portion 124c allows the user to grasp that the ilium should be supported by the ilium support portion 124c. The second concave portion 124b can be used as a marker for the ilium support portion 124c.

As illustrated in FIGS. 10, 11, and 12, the cushion 101 includes the lumbar support 120 and the cushion portion 110 that is located below the lumbar support 120 and has a seating portion 111 on which a user sits. The cushion 101 includes the lumbar support 120. Thus, similar effects to the lumbar support 120 described above can be achieved. The cushion 101 includes the cushion portion 110 that is located below the lumbar support 120 and has the seating portion 111. Since the back and waist of a user seated on the seating portion 111 can be firmly supported, the user's posture can be more reliably maintained in a satisfactory state.

For example, the posture detection system and the posture detection method according to the present disclosure can be applied to both the cushion portion 110 and the lumbar support 120. In other words, in the posture detection system and the posture detection method according to the present disclosure, similar to the cushions 100 and 200 described above, it is possible to detect the posture of a user seated on the cushion portion 110 and of a user wearing the lumbar support 120. Moreover, the shape, size, number, material, and arrangement of each part of the cushion portion and the lumbar support are not limited to the above examples, and can be appropriately modified.

The description above pertains to the lumbar support 120 provided with the side portion 123, which includes the ilium-facing portion 124 and the rib support portion 125. However, the lumbar support may also have a side portion that does not have at least one of the ilium-facing portion 124 and the rib support portion 125. The description above pertains to the lumbar support 120 that is an M-shape in a plan view. However, the lumbar support may not be an M-shape in a plan view, and the shape of the lumbar support can be appropriately modified. For example, the lumbar support may also lack at least one of the first concave portion 121c and the second concave portion 124b.

For example, the description in the embodiment mentioned above is given on the cushion 101 that includes the cushion portion 110 and the lumbar support 120 detachable from the cushion portion 110. However, the cushion may also be a cushion in which the cushion portion 110 and the lumbar support 120 are integrated. The cushion may be a cushion in which the cushion portion 110 and the lumbar support 120 are integrally molded.

In the above, the description is given on the example in which the cushion portion 110 includes the seating portion 111 and the lumbar support portion 112, and the seating portion 111 includes the buttock support portion 113 and the thigh support portion 114. However, the configuration of the cushion portion connected to the lumbar support according to the present disclosure can be appropriately modified within the spirit and scope of the invention described above.

Reference Signs List

| | |
|---|---|
| 1 | Core material |
| 1b | Lower surface |
| 2 | Seating portion |
| 2b | Ventilation hole |
| 3 | Lumbar support portion |
| 4 | Buttock support portion |
| 4b | Upper surface |
| 5 | Thigh support portion |
| 5b | Concave portion |
| 6 | Ischial support portion |
| 6b | Concave portion |
| 6c | Through-hole |
| 6f | Tapered surface |
| 7 | Inclined surface |
| 8, 9 | Convex portion |
| 9c | Inclined portion |
| 10 | General portion |
| 11 | Sacral support portion |
| 11d | Protruding surface |
| 11f | Recessed portion |
| 11g | Bottom surface |
| 11h | Inner surface |
| 20 | Fabric (inner fabric) |
| 22 | Outer fabric |
| 30 | Posture detection system |
| 31 | Seating portion sensor |
| 32 | Sacral support portion sensor |
| 33 | Thigh support portion sensor |
| 34 | Cable |
| 35 | Communication unit |
| 40 | Control unit |
| 41 | Seating determination unit |
| 42 | Posture determination unit |
| 43 | Leg-crossing determination unit |
| 50 | Output unit |
| 51 | Display unit |
| 51b | Seating time period |
| 51c, 51d, 51f, 51g, 51h | Time period |
| 51j | Time chart |
| 51k | Comment |
| 51p | Ranking |
| 52 | Alarm unit |
| 60 | Posture detection system |
| 70 | Fabric |
| 100, 200 | Cushion |
| 101 | Cushion |
| 110 | Cushion portion |
| 111 | Seating portion |
| 111b | Ventilation hole |
| 112 | Lumbar support portion |
| 113 | Buttock support portion |
| 114 | Thigh support portion |
| 115 | Ischial support portion |
| 116, 117 | Convex portion |
| 119 | Sacral support portion |
| 119b | Protruding surface |
| 119c | Recessed portion |
| 120 | Lumbar support |
| 121 | Lumbar spine support portion |
| 121b | Protrusion |
| 121c | First concave portion |
| 122 | Connection portion |
| 123 | Side portion |
| 123b | Inner surface |
| 124 | Ilium-facing portion |
| 124b | Second concave portion |
| 124c | Ilium support portion |
| 125 | Rib support portion |
| 126 | Top portion |
| A1, D1 | First direction |
| A2, D2 | Second direction |
| A3, D3 | Third direction |
| L1, L2 | Extension line |
| P | Intersection point |
| S | Mounting surface |
| T | Information terminal |
| θ1 | Intersection angle |
| θ2 | Inclination angle |

1. A posture detection system for detecting a posture of a user seated on a cushion, the cushion comprising: a seating portion having a buttock support portion on which a buttock of the user is placed; and a lumbar support portion having a sacral support portion configured to support a sacrum of the user from behind, the posture detection system comprising:
a seating portion sensor attached to the seating portion and configured to detect whether or not the user is seated on the seating portion;
a sacral support portion sensor attached to the sacral support portion and configured to generate an output based on one or more points of contact associated with the posture of the user seated on the seating portion;
a processing device configured to determine that the posture of the user is unsatisfactory based, at least in part, on the output generated by the sacral support portion sensor; and
an alarm configured to output an alarm in response to the processing device determining that the posture of the user is unsatisfactory.

2. The posture detection system according to claim 1, wherein
the sacral support portion sensor is configured to generate the output signifying that a threshold pressure is detected when the sacrum of the user is in contact with the sacral support portion,
the processing device determines that the posture of the user is unsatisfactory when the sacral support portion sensor fails to generate the output over a predetermined period of time, and
the alarm signifies that the sacrum of the user is not in contact with the sacral support portion.

3. The posture detection system according to claim 1, wherein
the output generated by the sacral support portion sensor measures a load applied by the sacrum of the user on the sacral support portion sensor,
the processing device determines that the posture of the user is unsatisfactory in response to determining that the load measured by the sacral support portion sensor is equal to or greater than a predetermined threshold, and
the alarm signifies that the posture of the user is leaning backward excessively.

4. The posture detection system according to claim 1, wherein
the seating portion includes a pair of thigh support portions aligned along a left-to-right direction as viewed by the user seated on the seating portion, and
the posture detection system further comprises a pair of thigh support portion sensors attached to the pair of thigh support portions, respectively, and configured to detect a load applied by one or more thighs of the user seated on the seating portion.

5. The posture detection system according to claim 1, further comprising a fabric configured to cover at least a part of a core material of the cushion, with the seating portion sensor and the sacral support portion sensor attached to the fabric.

6. The posture detection system according to claim 5, further comprising an outer fabric that covers the core material,
wherein the fabric is an inner fabric covered by the outer fabric.

7. The posture detection system according to claim 4, wherein
the core material of the cushion includes a concave portion formed on an upper surface of the seating portion and a concave portion formed on an upper surface of the thigh support portion, and
the seating portion sensor is embedded in the concave portion of the seating portion, and the thigh support portion sensor is embedded in the concave portion of the thigh support portion.

8. The posture detection system according to claim 1, wherein
the sacral support portion includes a protruding surface with a forward-protruding curved shape and a recessed portion that is indented rearward from the protruding surface, and
the sacral support portion sensor is attached to the recessed portion of the sacral support portion.

9. The posture detection system according to claim 4, further comprising a fabric configured to cover at least a part of a core material of the cushion,
wherein the fabric is an inner fabric to which the seating portion sensor, the sacral support portion sensor, and the thigh support portion sensor are attached.

10. The posture detection system according to claim 5, wherein
the fabric covers only a part of the core material, and
the fabric is fixed to the core material in such a way as to cover the seating portion and the sacral support portion.

11. The posture detection system according to claim 4, wherein
the seating portion extends in a first direction and a second direction, the first direction being a front-to-rear direction as viewed by the user seated on the seating portion and the second direction being a left-to-right direction as viewed by the user seated on the seating portion, and
the posture detection system includes:
the two seating portion sensors arranged along the second direction;
the sacral support portion sensor; and
the pair of thigh support portion sensors arranged along the second direction.

12. The posture detection system according to claim 1, wherein
the buttock support portion includes a pair of ischial support portions configured to support an ischium of the user seated on the seating portion, and
the seating portion sensor is arranged around each of the paired ischial support portions.

13. (canceled)

14. (canceled)

15. The posture detection system according to claim 4, wherein the processing device is configured to:
calculate a difference in loads applied to the pair of thigh support portions as measured by the pair of thigh support portion sensors; and
determine that the posture of the user is unsatisfactory in response to determining that the difference in loads measured by the pair of thigh support portion sensors over a predetermined period of time is equal to or greater than a predetermined threshold.

16. The posture detection system according to claim 4, wherein the processing device is configured to:
calculate an absolute value of a difference between a load applied to one of the thigh support portion sensors by a left thigh and a load applied to the other thigh support portion sensor by a right thigh; and
determine that the posture of the user is unsatisfactory in response to determining that the calculated absolute value is equal to or greater than a predetermined threshold, and
wherein the alarm signifies that a left-to-right balance of the user seated on the seating portion is out of balance.

17. The posture detection system according to claim 4, wherein
the processing device determines that the posture of the user is unsatisfactory when the pair of thigh support portion sensors only detects the load applied on one of the pair of thigh support portions for a predetermined period of time, and
the alarm signifies that the legs of the user are crossed.

18. The posture detection system according to claim 1, further comprising:
a display configured to display,
as a time chart, at least one of a seating time period during which the user is seated on the seating portion, a time period during which the posture is proper, a time period during which the posture is hunched, a time period during which the posture is excessively leaning back, a time period during which a left-to-right balance is unsatisfactory, and a time period during which legs of the user are crossed.

19. (canceled)

20. (canceled)

21. A posture detection system for detecting a posture of a user seated on a cushion, the cushion comprising a sacral support portion configured to support a sacrum of the user, the posture detection system comprising:
one or more sensors configured to detect one or more points of contact associated with the posture of the user seated on the cushion, including a sacral support portion sensor configured to generate an output in response to detecting when the sacrum of the user is in contact with the sacral support portion; and
a processing device configured to:
determine that the posture of the user is unsatisfactory based, at least in part, on the output generated by the sacral support portion sensor; and
generate an alarm in response to determining that the posture of the user is unsatisfactory

22. The posture detection system according to claim 21, wherein
the sacral support portion sensor is configured to generate the output signifying that a threshold pressure is detected when the sacrum of the user is in contact with the sacral support portion, and
the processing device is configured to:
determine that the posture of the user is unsatisfactory when the sacral support portion sensor fails to generate the output over a predetermined period of time; and
generate the alarm signifying that the sacrum of the user is not in contact with the sacral support portion.

23. The posture detection system according to claim 21, wherein
the output generated by the sacral support portion sensor measures a load applied by the sacrum of the user on the sacral support portion sensor, and
the processing device is configured to:
determine that the posture of the user is unsatisfactory in response to determining that the load measured by the sacral support portion sensor is equal to or greater than a predetermined threshold; and
generate the alarm signifying that the posture of the user is leaning backward excessively.

24. The posture detection system according to claim 21, wherein
the cushion comprises a pair of thigh support portions,
the one or more sensors include a pair of thigh support portion sensors configured to measure a load applied to one or both of the pair of thigh support portions, and
the processing device is configured to:
calculate a difference in loads applied to the pair of thigh support portions as measured by the pair of thigh support portion sensors; and
determine that the posture of the user is unsatisfactory in response to determining that the difference in loads measured by the pair of thigh support portion sensors over a predetermined period of time is equal to or greater than a predetermined threshold.
